(19) United States
(12) Patent Application Publication
(10) Pub. No.: US 2022/0040228 A1
(43) Pub. Date: Feb. 10, 2022

(54) MATERIALS AND METHODS FOR STOPPING RESPIRATORY VIRUS INFECTIONS

(71) Applicant: Frank Gaertner, San Diego, CA (US)

(72) Inventor: Frank Gaertner, San Diego, CA (US)

(21) Appl. No.: 17/444,809

(22) Filed: Aug. 10, 2021

Related U.S. Application Data
(60) Provisional application No. 63/063,736, filed on Aug. 10, 2020.
(60) Provisional application No. 63/069,481, filed on Aug. 24, 2020.
(60) Provisional application No. 63/088,779, filed on Oct. 7, 2020.

(51) Int. Cl. A61K 33/30; A61K 36/185; A61K 31/07; A61K 31/375; A61K 31/593; A61K 31/355; A61K 31/675; A61K 31/714; A61K 31/519; A61K 33/04; A61K 36/28; A61K 31/255; A61P 31/12

(52) U.S. Cl. CPC A61K 33/30; A61K 36/185; A61K 31/07; A61K 31/375; A61K 31/593; A61K 31/355; A61P 31/12; A61K 31/714; A61K 31/519; A61K 33/04; A61K 36/28; A61K 31/255; A61K 31/675

(57) ABSTRACT

The subject invention pertains to the use of zinc, zinc ionophore, one or more vitamins, folate, selenium, garlic, and/or Echinacea as a treatment of viral infections, and the training needed for subjects to recognize such symptoms to be able to stop respiratory viral infections including colds flu and coronavirus, or to prevent respiratory virus infections, such as SARS-CoV-2 infections. The subject invention further pertains to pharmaceutical compositions, packaged dosage formulations, and kits for stopping respiratory virus infections, or for preventing respiratory virus infections in contact subjects that might have such infections.

### CROSS-REFERENCE TO RELATED APPLICATIONS

This application claims the benefit of U.S. Provisional Application Ser. No. 63/063,736, filed Aug. 10, 2020; 63/069,481, filed Aug. 24, 2020; and 63/088,779, filed Oct. 7, 2020; all of which are hereby incorporated by reference in their entirety including any tables, figures, or drawings.

### BACKGROUND OF THE INVENTION

Respiratory viruses include RNA viruses that infect the upper and lower respiratory systems. Examples include influenza virus, respiratory syncytial virus (RSV), parainfluenza virus (PIV), human metapneumovirus (hMPV), coronavirus (CoV), and rhinovirus. Respiratory viruses are a leading cause of morbidity and mortality worldwide. Children, elderly, immunocompromised, and chronically ill individuals are at the greatest risk. Respiratory viruses infect the respiratory tract and are most commonly transmitted through the air.

The influenza virus is a negative-sense RNA virus. There are four classifications of the influenza virus—A, B, C, and, D—all of which, except D, are known to infect humans. The influenza virus is closely related to PIV, which is a common cause of respiratory infections in children, such as croup. Both influenza and PIV can cause mild to severe symptoms that often include fever, sore throat, muscle and joint pain, headache, coughing, and malaise.

RSV is an enveloped virus that contains a linear negative-sense RNA genome. It infects the cells of the mucosa, causing the infected mucosal cells to fuse together to form a syncytium. The incubation period is 2-8 days and often displays mild symptoms similar to common colds; however, wheezing and asthma are more common among individual who were infected by a RSV early in life. Like RSV, hMPV is a common cause of respiratory infection in young children. hMPV is a negative-sense single-stranded RNA virus. Both hMPV and RSV can cause mild symptoms such as those associated with the common cold but can also lead to severe disease in premature infants, immunocompromised persons, and older adults.

Coronaviruses are enveloped viruses of the family Coronaviridae, which are transmitted through the air and primarily infect the cells of the upper respiratory and gastrointestinal tract of mammals and birds. The name coronavirus is derived from the Latin “crown” or “halo”, which refers to its characteristic morphology, which resembles a crown or solar corona when imaged using an electron microscope.

Coronaviruses cause illness in adults and children ranging from the common cold to more severe diseases. Common signs of infection include respiratory symptoms, fever, cough, shortness of breath, and breathing difficulties. In more severe cases, coronavirus infection can cause pneumonia, severe acute respiratory syndrome (SARS), kidney failure, and even death. The widely publicized human coronavirus discovered in 2003, SARS-CoV, causes both upper and lower respiratory tract infections.

Coronaviruses are zoonotic, meaning they are transmitted between animals and humans. Several known coronaviruses are circulating in animals that have not yet infected humans. A novel coronavirus (nCoV) is a new strain that has not been previously identified in humans. There are currently no vaccines or antiviral drugs to prevent or treat human coronavirus infections.

Following the outbreak of SARS in 2003, which had begun the prior year in Asia, and secondary cases elsewhere in the world, the World Health Organization (WHO) issued a press release stating that a novel coronavirus, which as identified by a number of laboratories, was the causative agent for SARS. The virus was officially named the SARS coronavirus (SARS-CoV).

In September 2012, a new type of coronavirus was identified, initially called Novel Coronavirus 2012, and now officially named Middle East respiratory syndrome coronavirus (MERS-CoV). In May 2014, two United States cases of MERS-CoV infection were recorded, both occurring in healthcare workers who worked in Saudi Arabia and then traveled to the U.S. In May 2015, an outbreak of MERS-CoV occurred in the Republic of Korea, when a man who had traveled to the Middle East visited hospitals in the Seoul area to treat his illness, causing one of the largest outbreaks of MERS-CoV outside the Middle East.

In December 2019, a pneumonia outbreak was reported in Wuhan, China, and was traced to a novel strain of coronavirus, which was given the interim name 2019-nCoV by the World Health Organization (WHO), later renamed SARS-CoV-2 by the International Committee on

Taxonomy of Viruses (Zhu N. et al, “A Novel Coronavirus from Patients with Pneumonia in China, 2019”, N Engl J Med, Feb 2020, 382(8):727-733, Epub 24 January 2020). Coronavirus disease 2019 (COVID-19) is the infectious disease caused by SARS-CoV-2. SARS-CoV-2 has killed more people than 2003 SARS outbreak.

Rhinoviruses are single-stranded, positive sense RNA viruses that are usually spread through aerosols of respiratory droplets and from fomites. Rhinoviruses are the primary causative agents of the common cold, with symptoms that include sore throat, runny nose, nasal congestion, sneezing, and cough. There are three recognized serotypes of human rhinovirus, including A, B, and C. Each species of rhinovirus has dozens of recognized index numbers.

There remains a need for a safe and effective method of preventing, treating or as defined and described herein, stopping infections of respiratory viruses and associated symptoms, in particular those related to COVID-19, and its causative agent, SARS-CoV-2.

BRIEF SUMMARY OF THE INVENTION

The present invention concerns the use of zinc for the prevention, prevention, and specifically, as described herein, the stopping of respiratory virus infections, such as SARS-CoV-2. In some embodiments, the zinc is administered, preferably self-administered or administered by a qualified medical professional, with additional components including a zinc ionophore, vitamins, folate, selenium, garlic, and/or Echinacea. The zinc ionophore can be elderberry. The vitamins can include vitamin A, vitamin C, vitamin D₃, vitamin E, vitamin B₆, and vitamin B₁₂.

One aspect of the invention is a method for the treatment or prevention of respiratory virus infection, comprising administering zinc to a human subject in need thereof. In some embodiments, the zinc is administered to a human subject at the outset of an infection by a respiratory virus, such as SARS-CoV-2, as therapy therapeutic means for stopping the infection by inactivating the virus's replication at the initial site of infection and at the earliest possible moment following said infection. In some embodiments, the subject has the disease COVID-19. In other embodiments, the zinc is administered in high-dose capsular or other form in common use by a qualified medical practitioner to a human subject not infected by a respiratory virus, such as SARS-CoV-2, as prophylaxis (to prevent or delay the onset of a respiratory virus infection).

Another aspect of the invention concerns, in its broadest sense, a method for experimentally inhibiting or, as described herein, stopping a respiratory virus infection, such as SARS-CoV-2, in a human cell, comprising contacting the cell experimentally in vitro or therapeutically in vivo with zinc, before or after the cell is infected.

Another aspect of the invention concerns a method for a medical professional to treat symptoms or consequences of a respiratory virus infection, comprising administering zinc to a human subject in need thereof. The symptoms may be what have been described as a cytokine storm, which can lead to further consequences such as, for example fibrosis of tissues, including lung tissues. The pathophysiology of SARS-CoV-2-induced acute respiratory distress syndrome (ARDS) has similarities to that of severe community-acquired pneumonia caused by other microorganisms. The overproduction of early response proinflammatory cytokines (tumor necrosis factor (TNF), IL-6, and IL-1(3) results in the cytokine storm, leading to an increased risk of vascular hyperpermeability, multi-organ failure, and death.

In some embodiments of the methods of the invention, the respiratory virus is an influenza virus, a PIV, a RSV, hMPV, a coronavirus, and/or a rhinovirus.

In some embodiments of the methods of the invention, the influenza virus can be influenza A, influenza B, influenza C, or influenza D. Influenza A can be H1N1, H2N2, H3N2, H5N1, H7N7, H1N2, H9N2, H7N2, H7N3, H10N7, H7N9, or H6N1.

In some embodiments of the methods of the invention, the RSV can be RSV-A with genotypes GA1, GA2, GA3, GA4, GA5, GA6, GA7, SAA1, NA1, or NA2 or RSV-B with genotypes GB1, GB2, GB3, GB4, SAB 1, SAB2, SAB3, BA1, BA2, BA3, BA4, BAS, or BA6

In some embodiments of the methods of the invention, the PIV can be HPIV-1, HPIV-2, HPIV-3, or HPIV-4.

In some embodiments of the methods of the invention, the hMPV can be from the A1, A2, B1, or B2 lineage.

In some embodiments of the methods of the invention, the human coronavirus is selected from among SARS-CoV-2, SARS-CoV, and MERS-CoV.

In some embodiments of the methods of the invention, the human coronavirus is a common human coronavirus, such as type 229E, NL63, 0C43, and HKU1.

In some embodiments of the invention, the rhinovirus is a common rhinovirus, such as serotypes A, B, or C.

Another aspect of the invention concerns a composition comprising zinc for treatment of respiratory virus infections, such as a SARS-CoV-2 infection. In some embodiments of the composition, the composition further comprises a zinc ionophore, vitamins, folate, selenium, garlic, and/or Echinacea. The vitamins can include vitamin A, vitamin C, vitamin D₃, vitamin E, vitamin B₆, vitamin B₁₂. In certain embodiments, the zinc ionophore is elderberry. In some embodiments of the composition, the composition comprises a packaged dosage formulation or a kit for treatment or prevention of a respiratory virus infection.

In certain embodiments, a person in need can initiate self-treatment within about 24 hours to about 48 hours of the time symptoms of viral infection commence and/or when a person has come in contact with an infected person, as a prophylactic treatment.

Another aspect of the invention concerns kits comprising, in one or more containers, zinc of the present invention, preferably a small, single-dose, easy-to-carry, rapid-application kit. A kit of the invention can also comprise one or more other compounds, biological molecules, or drugs. In one embodiment, the kit of the invention comprises zinc, and optionally comprises one or more of a composition used in treating a viral infection (e.g., a human coronavirus infection, such as SARS-CoV-2) that can comprise a zinc ionophore, vitamins, folate, selenium, garlic, and/or Echinacea.

### DETAILED DISCLOSURE OF THE INVENTION

The invention disclosed herein could be one of the most important inventions of our time. Yet, it consists of nothing more than a simple composition of readily available components that can be self-administered or delivered by assistance at certain times and in specific ways. Nonetheless, simple though the invention may be, the significance of its disclosed compositions and methods could make life versus death differences to an individual's health and make similar differences globally once is the invention becomes widely known. Although the present invention is simple, it is clearly important, and yet, there can be no question, the life-saving opportunities presented by the present invention have been missed until now. To lend emphasis to the latter statement, if those opportunities had been realized, it is possible we would not now be in the throes of a socially devastating, life threatening pandemic.

The present invention offers the realization of a dream, a dream that ordinary people possessing inexpensive, easy to obtain, easy to prepare and easy to use materials and methods that could have the ability to protect themselves from the debilitating effects of colds, flu, and coronavirus. But even though the present invention shows that that dream is not impossible to realize, and even though that dream is now more important and timely than ever due to the pandemic, that dream continues to await realization. It simply might be that the dream was thought to be an impossible one. Possibly, but a more relevant reason may be due to the dream's special self-administering properties and an overlooked requirement.

In order for the present invention to work, subjects need to be self-administering trained to pay very close attention to noticing the faint, barely detectable, initial symptoms that are produced by all respiratory viruses when the viruses first begin to infect a subject's body. Therefore, without training, it is likely that most individual users, even if they had inadvertently come up with potentially helpful versions of the method of the present invention, would not have recognized the importance of the faint symptoms associated with such respiratory viral attacks. In that case, no matter how well such methods might have worked to stop such a viral attack, there would have been little chance for the products to work in the viral-stopping mode that is characterized by the present invention. Also, without understanding the prime importance of the faint, initial signals of infection produced by a respiratory virus, even dedicated researchers testing the efficacy of available, self-administered products may have missed the opportunity to discover any ability such products might have had to stop respiratory viral infections. That is, instead of focusing on the faint, initial symptoms, said researchers likely focused on serious symptoms of respiratory virus disease. Having done so, such researchers would have, naturally, turned their attention to preventing serious symptoms with vaccines or prophylactic healthcare plans or remediating disease symptoms with drug therapies or high technology cures.

Thus, the method of, the present invention is distinct from all other respiratory virus treatment inventions in that it focuses on users that are trained to self-administer treatment based on early, faint, initial symptoms of respiratory viral infections with products designed to stop all such infections at their onset, wherein the products so designed are a specific set of readily available, inexpensive, nonprescription, off-the-shelf products delivered in a specific, well-defined, and effective way.

Based on the method, an infected subject is to pay close attention to and immediately act on any and all faint, barely noticeable symptoms, and they are to act on those faint symptoms, with subject method's safe to use, readily available, inexpensive minerals, vitamins and supplements, which are components that can interfere with viral replication and can enhance an individual's immunity. The compositions of the method described herein are to be initiated at a time that strives to be as coincident as possible with the respiratory virus's entrance into the human body. The specific method to arrive as closely as possible to this coincidence is, herein, described in detail.

To summarize, it is the premise of the disclosed invention that all RNA respiratory virus infections can be stopped by alert, early treatment of said viral infections with readily available and easily practiced compositions and methods. To that end, the compositions and methods of the present invention have been logically assembled and devised based on interpretation of confirmed scientific data. For example, respiratory viruses primarily enter a subject's body by way of the subject's olfactory system and it is in this area of the subject's body where a respiratory virus's invasion of mucosal cells produces its initial, faint, irritating signals of attack. It's also important to realize that it is at this initial time and in this place in the subject's body that viral particles are still limited in number, most exposed and, therefore, most vulnerable to the self-treatment methods described herein.

Zinc ions can inhibit the replicase polymerase of RNA/RNA respiratory virus as well as the transcriptase polymerase of RNA/DNA respiratory virus. In addition, zinc is an important element in the functioning of the human immune system as it facilitates antibody-producing lymphocytes, activates human immune system first-responders such as killer T-cells and activates virus killing cytokines such as interferon-gamma. Also, zinc ionophores are needed for intra and intercellular function of positively charged zinc ions. Such ionophores are found in food, good examples of which include quercetin and green tea. However, based on personal experience and popularity of the protective additive, the ionophore isolated from extracts of Elderberry has been chosen to be well suited for facilitating the entry of virus-inactivating zinc ions into infected cells. Also, a number of immune-system-enhancing additives such as garlic allicin, Echinacea, vitamins A, B, C, D, and E, folate and selenium are included in the composition and methods of the subject invention. But, knowing all of these things is not sufficient to stop RNA respiratory virus infections.

Based on the above knowledge, the treatments needed to stop the progress of a flu, cold or coronavirus disease, must be applied appropriately, without delay and at the earliest possible moment subsequent to an invasion of a subject's olfactory system by a respiratory virus. This important aspect of the invention can only be accomplished by a subject paying very close attention to the possibility of propagation of such a respiratory virus by the subject sensing it or thinking that the subject senses it, and then immediately addressing, the very faint irritations occurring in the mucosa of the subject's olfactory system or other unexpected slight discomforts occurring elsewhere in the subject's body. For example, most often, but not always, such an attack can be recognized to be occurring at the back of a subject's throat as a very faint scratchy or ticklish sensation just behind the subject's uvula.

Such faint symptoms can be hard to notice, especially when they are in sharp contrast to those normally associated with the initial symptoms of a viral disease such as COVID-19. For example, with a disease as dangerous as COVID-19, the easily noticed, hard-to-miss symptoms normally thought of as the initial symptoms of the disease include dry cough, extreme tiredness, fever, shaking chills, throbbing headaches, tiredness, overall pain, nausea, vomiting, and shortness of breath. Whereas, the barely detectable early symptoms that are significant to the success of the present invention include very faint scratchy or ticklish throat irritations, a sudden snotty nose, sudden minor congestion, sudden feelings of a slight wobble in body temperature, sudden minor aches, sudden minor eye irritation, and/or overall, sudden, faint sickish feeling. These faint, barely noticeable symptoms can all be false alarms such as symptoms that are unrelated to viral disease attacks or symptoms that are simply the products of one's imagination. But that is where this invention for the self-treatment of colds, flu, and coronavirus disease stands apart from other therapies of this type. It simply does not matter if the noticed symptoms are false alarms. The invention is robust enough and safe enough to use on all false alarms. In fact, subjects are encouraged to act on any suspicion or reason that they think they might “have something”.

The invention involves self-treatments and, if done as described, the self-delivered treatments, can be safely done and leave the subject in good health, without having a need for help from an emergency room doctor or the need for an ICU life-support system. Because of the dangerous diseases the treatments of the invention are designed to address, one does not want to take chances.

Although the above uncertainty may create credibility issues for anyone claiming they have successfully defeated a respiratory virus, specifically, referenced here for convenience as an Upper Respiratory Virus (URV disease), it really does not matter why a subject stays well by the administration of the invention's treatments. However, an individual using the method may know why they are staying well when others in contact with the subject are getting sick and he/she is not.

If replication of an invading respiratory virus can be eliminated by an entire population paying close attention to early symptoms that are treated early with the methods disclosed herein, the devastation wrought by COVID-19 and other RNA respiratory viruses on such a population could be expected to be eliminated from that population far into the future. But, again, this will only happen if subjects are well trained to stay alert and use treatments such as those described herein. To repeat, if a subject feels a slight scratchiness in their throat, body temperature fluctuation or other sickish feeling, they are not to wait. They are to be trained to act without hesitation. For this training a brief training manual in the form of a URV Symptom Data Record can be employed as exemplified in this disclosure.

An important consideration, heretofore neglected, is the important difference that exists between prophylaxis and the stopping of an RNA respiratory virus infection. For example, one could on a daily basis prophylactically take the first treatment of the first round of the URV method as described below. The effect of such a prophylactic treatment might, indeed, ward off a URV attack on the morning the treatment was used. If such a condition were to cause the subject to wait, it is unclear whether that wait would cause the subject to notice the viral invasion in time to stop the infection. In the specific instance of a subject being in known contact with a viral-infected person, heightened awareness by the contacted subject in addition to multiple prophylactic treatments with the disclosed invention and done as if the subject had experienced faint symptoms over multiple days as illustrated in this disclosure could be done as a reasonable, protective precaution.

The following is an example using the invention's centerpiece method for stopping a URV infection, a method we have chosen to call the URV2020. Ideally, subjects sensing a possible respiratory attack immediately take, in the manner described in detail below for the URV2020, the full first treatment of Round 1 of the method. If after the first treatment, the subject feels no more symptoms, they need do nothing more. But to be safe they can always take a full second treatment of Round 1 as a precautionary treatment, and then if they still feel nothing untoward, after that do nothing more. However, if symptoms persist or worsen, a subject is to be taught to proceed and complete the four treatments of Round 1 as described. The goal is to aggressively attack the virus when it first attacks to discharge it from a subject's system before more serious symptoms have had a chance to develop.

Optimally, a subject will eliminate the virus and its symptoms in less than 12 hours after having used the four treatments of Round 1. However, if symptoms persist, multiply or worsen, the subject is to proceed with all four treatments of Round 2. These four treatments are identical to the four treatments of Round 1 except that the zinc concentration has been reduced from 18.3-mg to just 5-mg in each of the four treatments of Round 2 in order to avoid an overall treatment that would exceed the advised limit of 100-mg of zinc used within a 24-hour period. However, it may develop that medical professionals skilled in the art may want sick patients to take 1000-mg or more of zinc to remediate symptoms of an ongoing respiratory viral disease, but to be clear remediation is, primarily, not what the present invention is about. The invention's primary focus is stopping a virus infection before its more noticeable, serious symptoms have had a chance to develop.

The combination of orally dissolving a zinc lozenge with three Elderberry-zinc-ionophore-dissolvable-tablets is a new method and the basis of a new composition of matter.

The addition of an Elderberry capsule with two garlic tabs and two Echinacea capsules is an additional new method and the basis for a new composition of matter. Recognizing early symptoms combined with the use of the new methods, compositions of matter and training of subjects to recognized the early symptoms of RNA respiratory viral attacks represent new ways to use readily available commercial products and represent an opportunity for the manufacture of improved, new composite, compositions of matter and improved delivery methods designed to defeat all RNA respiratory virus infections in even less time than 24 hours. However, there is currently no home remedy, or other remedy, that even suggests what the present invention is designed to accomplish, the stopping of all cold, flu and coronavirus infections in less than 24 hours, as well as an end to the 2019 pandemic.

The following example of Training Data Records illustrate the way the URV2020 method can be used to test if symptom are the real thing or false alarms and they exemplify the use of precautionary treatments, which are an especially good idea in this time of the COVID-19 pandemic and the uncertainty that one would have involving necessary-to-treat early symptoms. Table 1 shows the Training Data Record prior to its use.

TABLE 1
URV2020 24-Hr Symptom-Data-Trainer
Date _ _: _ _ : _ _ _ _, Time of onset _ _: _ _ am/pm (circle one)
Zip Code _____; Age___: coded ID ____: Estimate health prior to onset ________.
Use ~° F. for Fever, Y for Yes, and a Blank for No. The chart starts at Hour One of
suspected disease onset. Record results for each of the following 23 hours after that.
Use new forms for any reports after hour 24. Share below known cold, flu, or
COVID19 contacts + any events not included in the chart.

| Hour + | | | | | | | | Fever | | |
|---|---|---|---|---|---|---|---|---|---|---|
| military | S | T | C | S | S | Sore | | in | | Remedy |
| Time | T | W | M | N | F | throat | Cough | ~° F. | Ache | Used* |
| 1 | | | | | | | | | | |
| 2 | | | | | | | | | | |
| 3 | | | | | | | | | | |
| 4 | | | | | | | | | | |
| 5 | | | | | | | | | | |
| 6 | | | | | | | | | | |
| 7 | | | | | | | | | | |
| 8 | | | | | | | | | | |
| 9 | | | | | | | | | | |
| 10 | | | | | | | | | | |
| 11 | | | | | | | | | | |
| 12 | | | | | | | | | | |
| 13 | | | | | | | | | | |
| 14 | | | | | | | | | | |
| 15 | | | | | | | | | | |
| 16 | | | | | | | | | | |
| 17 | | | | | | | | | | |
| 18 | | | | | | | | | | |
| 19 | | | | | | | | | | |
| 20 | | | | | | | | | | |
| 21 | | | | | | | | | | |
| 22 | | | | | | | | | | |
| 23 | | | | | | | | | | |
| 24 | | | | | | | | | | |

*1. Pint of Water; 2. Cold Eeze lozenge; 3. Three Zarbees pills; 4. Elderberry capsule; 5. Two Garlic tabs; 6. Two Echinacea
Early symptoms: ST = scratchy throat, TW = temperature wobble, CM = cranky mood, SN = snotty nose, SF = sickish feeling
For each hour show by numerical ID the products used. Record below any issues, or special results. Circle if in contact: COLDS, FLU, or COVID19. If symptoms persist beyond Hour 4, act as though you have something serious. Continue treatments approximately as follow: Hour 1: 123456; Hour 2: 123456; Hour 3: 123456; Hour 4; 123456: Hour 8: 13456 Hour 9: 13456 Hour 10: 13456; Hour 11: 13456 (Note: zinc lozenge is left out starting at Hour 8). If symptoms persist beyond 24 hours, record results on a 2^{nd} copy of this form, and get medical help if necessary. If you take prescription drugs, talk to your doctor about contraindications. For example, zinc can interfere with thyroid medication.

An aspect of the invention concerns a method for treatment or prevention of a respiratory virus infection, such as SARS-CoV-2, in a human subject, comprising administering to the human subject an effective amount of zinc, one or more zinc ionophores, vitamins, folate, and/or selenium. The zinc, one or more zinc ionophores, vitamins, folate, and/or selenium may be administered to or, preferably, administered by the human subject before or after initiation of the respiratory virus infection, thereby treating the respiratory virus infection. In some embodiments, the subject has the respiratory virus infection, such as COVID-19, at the time that zinc is administered. In some embodiments, the subject administers treatment after having noticed the first noticeable, effects of a respiratory virus infection. The slight irritation will most commonly be noticed to take place at the back of a subject's throat, and it is this time and place that is coincident with the optimal time and place for the zinc and supportive products such as the zinc ionophore to be first administered.

In certain embodiments, the zinc and zinc ionophore can be administered concurrently with other agents, such as, garlic, and/or Echinacea. Additionally, the one or more vitamins, folate, selenium, garlic, and Echinacea can be administered before or after administration of zinc.

In certain embodiments, the zinc, preferably in lozenge and/or tablet form, can be administered, preferably by a physician, after the viral infection with the hope of limiting or preventing complications or serious symptoms of the previously acquired infection.

In certain embodiments, the zinc can be administered, preferably, by a qualified medical practitioner, to treat symptoms, complications, or comorbidities of a respiratory virus infection. Complications, comorbidities, or symptoms can include a cytokine storm, or fibrosis of tissues, such as, for example, lung tissue.

Another aspect of the invention concerns a method for inhibiting and thereby stopping a respiratory virus infection in a human cell, comprising experimentally contacting the cell in vitro or in vivo with zinc, before or after the cell is infected and, optionally, with one or more zinc ionophores, vitamins, folate, selenium, garlic, and/or Echinacea.

The respiratory virus can be a RNA respiratory virus, an upper respiratory virus, and/or a lower respiratory virus.

The respiratory virus can be an influenza virus, a PIV, a RSV, hMPV, a coronavirus, and/or a rhinovirus.

The respiratory virus can be an influenza virus. The influenza virus can be influenza A, influenza B, influenza C, or influenza D. Influenza A can be H1N1, H2N2, H3N2, H5N1, H7N7, H1N2, H9N2, H7N2, H7N3, H1ON7, H7N9, or H6N1.

The respiratory virus can be a RSV. The RSV can be RSV-A with genotypes GA1, GA2, GA3, GA4, GA5, GA6, GA7, SAA1, NA1, or NA2 or RSV-B with genotypes GB1, GB2, GB3, GB4, SAB 1, SAB2, SAB3, BA1, BA2, BA3, BA4, BA5, or BA6

The respiratory virus can be a PIV. The PIV can be HPIV-1, HPIV-2, HPIV-3, or HPIV-4.

The respiratory virus can be a hMPV. The hMPV can be from the A2, A2, B1, or B2 lineage.

The respiratory virus can be a human upper/lower respiratory virus, such as a coronavirus. The human coronavirus may be any kind or subgroup, including alpha, beta, gamma, and delta. In some embodiments of the aforementioned methods of the invention, the human coronavirus is selected from among SARS-CoV-2, SARS-CoV, and MERS-CoV. In some embodiments of the aforementioned methods of the invention, the human coronavirus is a common human coronavirus, such as type 229E, NL63, 0C43, and HKU1.

The respiratory virus can be an upper/lower respiratory virus, such as a rhinovirus. The rhinovirus is a common rhinovirus, such as serotypes A, B, or C.

Another aspect of the invention concerns a composition comprising zinc and, optionally, with one or more zinc ionophores, vitamins, folate, selenium, garlic, and/or Echinacea.

### Zinc and Zinc Ionophores

In certain embodiments, zinc and a zinc ionophore are administered to a human subject or are in a composition. The zinc can be formulated into pharmaceutically acceptable salt forms or hydrate forms, such as zinc gluconate, zinc oxide, zing citrate, or zinc glycinate.

Zinc can interfere with RNA dependent, RNA polymerase. The zinc ionophore, such as Elderberry, blueberry, green tea, quercetin, and quinine, can help zinc enter infected cells to inactivate the virus as it replicates in the human subject, particularly in the nose, nasal passage, throat, trachea, or mouth.

In certain embodiments, the zinc and zinc ionophore may increase antibody production and/or enhance lymphocyte function. Zinc can activate killer T-cell lymphocytes and cytokines such as interferon-gamma. Certain populations, including the elderly and immunocompromised, may become zinc deficient. The zinc administration of the subject invention can supplement zinc levels in the diets.

### Additional Additives

In certain embodiments, one or more of garlic, folate, selenium, Echinacea, and/or vitamins can be administered with zinc and/or one or more zinc ionophores for treatment of a respiratory virus infection or as a prophylactic treatment. The vitamins can be one or more of vitamin A, vitamin C, vitamin D₃, vitamin B₆, and/or vitamin B₁₂. Separately, interleukin-7 may be administered, preferably by a qualified medical practitioner. Interleukin-7 can increase killer T-cells levels.

In certain embodiments, the garlic, folate, selenium, Echinacea, N-acetyl cysteine, melatonin, and/or vitamins can be used by a qualified medical practitioner to treat co-morbidities, complications, symptoms, or other consequences of a respiratory virus infection. Melatonin and N-acetyl cysteine, a precursor to glutathione, can reduce a cytokine storm. Examples of complications include a cytokine storm, pneumonia, viral sepsis, acute respiratory distress syndrome, kidney failure, fibrosis of tissues, and cytokine release syndrome. Examples of symptoms that can be avoided by early treatment with the subject method and compositions include fever, cough, fatigue, shortness of breath, and loss of smell and/or taste.

### Compositions and Treatment

The zinc and/or a zinc ionophore, garlic, folate, selenium, Echinacea, and/or vitamins of the present invention can be formulated into in a tablet, capsule, emulsion, hydrogel, solution, suspension, tincture, powder, lozenge, lotion, spray, drop, ointment, cream, or paste.

Administration of zinc and/or a zinc ionophore, garlic, folate, selenium, Echinacea, and/or vitamins can be carried out in the form of an oral tablet, capsule, or liquid formulation containing a therapeutically effective amount of zinc and/or a zinc ionophore, garlic, folate, selenium, Echinacea, and/or vitamins. Administration can be oral delivery and includes the possibility of formulations that can be prescribed and used with the intervention by a medical practitioner. One aspect of the subject invention relates to subjects self-administering safe amount of presently available commercial products in composition and manner heretofore unforeseen in the art. However, with the assistance of qualified medical practitioners, therapeutic or prophylactic application of the of zinc and/or a zinc ionophore, garlic, folate, selenium, manganese, Echinacea, and/or vitamins, and compositions containing them, can be accomplished by any suitable therapeutic or prophylactic method and technique presently or prospectively known to those skilled in the art. The zinc and/or a zinc ionophore, garlic, folate, selenium, manganese, Echinacea, and/or vitamins can be administered by any suitable route known in the art by a qualified medical practitioner including, for example, oral, intramuscular, intraspinal, intracranial, nasal, rectal, parenteral, subcutaneous, or intravascular (e.g., intravenous) routes of administration. Administration of the zinc and/or zinc ionophores of the invention can be continuous or at distinct intervals as can be readily determined by a person skilled in the art.

Zinc, a zinc ionophore, garlic, folate, selenium, Echinacea, and/or vitamins and compositions comprising them can be formulated according to known methods for preparing pharmaceutically useful compositions. Formulations are described in detail in a number of sources which are well known and readily available to those skilled in the art. For example, Remington's Pharmaceutical Science by E. W. Martin describes formulations that can be used in connection with the subject invention. In general, the compositions of the subject invention will be formulated such that an effective amount of the bioactive inhibitor is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art. Examples of carriers or diluents for use with the subject inhibitors include, but are not limited to, water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these. Formulations of the inhibitors can also comprise suspension agents, protectants, lubricants, buffers, preservatives, and stabilizers. To provide for the administration of such dosages for the desired therapeutic treatment, pharmaceutical compositions of the invention will advantageously comprise between about 0.1% and 45%, and especially, 1 and 15% by weight of the total of one or more of the inhibitor based on the weight of the total composition including carrier or diluent.

The subject invention also concerns a packaged dosage formulation comprising in one or more packages, packets, or containers at least one zinc, a zinc ionophore, garlic, folate, selenium, Echinacea, and/or vitamins, and/or composition of the subject invention formulated in a pharmaceutically acceptable dosage. The package can contain discrete quantities of the dosage formulation, such as tablet, capsule, tablets, lozenge, and powder. The package can preferably be in an easy-to-carry form and ready for immediate use.

The quantity of the zinc can vary from about 0.1 mg to about 100 mg, about 1 mg to about 75 mg, or about 5 mg to about 45 mg. In some embodiments, the amount is in the range of 5 mg to about 35 mg, to be administered, 1, 2, 3, 4, 5, 6, 7, or 8 times per day for 1 day or 2 days or for more days if necessary, the latter two or more days being done preferably under a qualified medical practitioner's supervision. The zinc can be formulated into pharmaceutically-acceptable salt forms, such as, for example, zinc gluconate and/or zinc oxide. The zinc can be delivered by commercially available compositions, such as, for example, Cold Eeze and/or Zarbee's Elderberry and Honey.

The quantity of the zinc ionophore, such as elderberry fruit extract, can vary from about 1 mg to about 5000 mg, about 10 mg to about 2000 mg, or about 100 mg to about 1000 mg. In some embodiments, the amount is in the range of about 200 mg to about 1000 mg, to be administered, 1, 2, 3, 4, 5, 6, 7, or 8 times per day for 1 day or 2 days or for more days if necessary, preferably under a qualified medical practitioner's supervision. The zinc ionophore can be delivered in the form of a fruit extract and/or juice. The elderberry can also be part of a blend, such as a 326 mg blend comprising honey, elderberry fruit extract, chicory, and baker's yeast beta glucan. The zinc ionophore, such as elderberry, can be delivered by commercially available compositions, such as, for example, Quantum, Inc. Elderberry, New Chapter Elderberry Force, and/or Zarbee's Elderberry and honey.

The quantity of the folate can vary from about 1 μg to about 5000 μg, about 10 μg to about 2000 μg, or about 100 μg to about 1000 μg. In some embodiments, the amount is about 666 μg, to be administered, 1, 2, 3, 4, 5, 6, 7, or 8 times per day for 1 day or 2 days or for more days if necessary, preferably under a qualified medical practitioner's supervision. The folate can be delivered by a commercially available composition, such as, for example, Zarbee's Elderberry and honey.

The quantity of the selenium can vary from about 0.1 μg to about 1000 μg, about 1 μg to about 200 μg, or about 5 μg to about 100 μg. In some embodiments, the amount is about 15 μg, to be administered, 1, 2, 3, 4, 5, 6, 7, or 8 times per day for 1 day or 2 days or for more days if necessary, preferably under a qualified medical practitioner's supervision. The selenium can be formulated into pharmaceutically-acceptable chelate forms, such as, for example, selenium amino acid chelate. The selenium can be delivered by commercially available compositions, such as, for example, Zarbee's Elderberry and honey.

The quantity of the Echinacea can vary from about 1 mg to about 5000 mg, about 10 mg to about 2000 mg, or about 100 mg to about 1200 mg. In some embodiments, the amount is about 800 mg, to be administered, 1, 2, 3, 4, 5, 6, 7, or 8 times per day for 1 day or 2 days or for more days if necessary, preferably under a qualified medical practitioner's supervision. The Echinacea can be delivered by a commercially available composition, such as, for example, Nature's Bounty Echinacea.

The quantity of the garlic can vary from about 1 mg to about 10000 mg, about 10 mg to about 7500 mg or about 100 mg to about 5000 mg. In some embodiments, the amount is about 4000 mg, to be administered, 1, 2, 3, 4, 5, 6, 7, or 8 times per day for 1 day or 2 days or for more days if necessary, preferably under a qualified medical practitioner's supervision. The garlic can be delivered by a commercially available composition, such as, for example, Nature's Bounty Garlic.

The quantity of the vitamins can vary from about 0.01 μg to about 5000 mg. The quantity of the vitamin A can vary from about 1 μg to about 5000 μg, about 10 μg to about 2000 μg, or about 100 μg to about 1000 μg. The vitamin A can be in the form of retinyl palmitate and/or retinyl acetate. In some embodiments, the amount is in the range of about 300 μg, to be administered, 1, 2, 3, 4, 5, 6, 7, or 8 times per day for 1 day or 2 days or for more days if necessary, preferably under a qualified medical practitioner's supervision. The vitamin A can be delivered by commercially available compositions, such as, for example, Zarbee's Elderberry and honey.

The quantity of the vitamin C can vary from about 1 mg to about 5000 mg, about 10 mg to about 2000 mg, or about 100 mg to about 1500 mg. In some embodiments, the amount is about 500 mg, to be administered, 1, 2, 3, 4, 5, 6, 7, or 8 times per day for 1 day or 2 days or for more days if necessary, preferably under a qualified medical practitioner's supervision. The vitamin C can be in the form of ascorbic acid, sodium ascorbate, potassium ascorbate, magnesium ascorbate, or other pharmaceutically-acceptable salt. The vitamin C can be delivered by commercially available compositions, such as, for example, Zarbee's Elderberry and honey.

The quantity of the vitamin D₃ can vary from about 0.1 μg to about 100 μg, about 1 μg to about 50 μg, or about 5 μg to about 30 μg. In some embodiments, the amount is about 20 μg, to be administered, 1, 2, 3, 4, 5, 6, 7, or 8 times per day for 1 day or 2 days or for more days if necessary, preferably under a qualified medical practitioner's supervision. The vitamin D₃ can be in the form of cholecalciferol. The vitamin D₃ can be delivered by commercially available compositions, such as, for example, Zarbee's Elderberry and honey.

The quantity of the vitamin E can vary from about 0.1 mg to about 1000 mg, about 1 mg to about 200 mg, or about 10 mg to about 100 mg. In some embodiments, the amount is in the range of about 15 mg, to be administered, 1, 2, 3, 4, 5, 6, 7, or 8 times per day for 1 day or 2 days or for more days if necessary, preferably under a qualified medical practitioner's supervision. The vitamin E can be in the form of d-a tocopheryl succinate. The vitamin E can be delivered by commercially available compositions, such as, for example, Zarbee's Elderberry and honey.

The quantity of the vitamin B₆ can vary from about 0.01 mg to about 1000 mg, about 0.1 mg to about 100 mg, or about 1 mg to about 10 mg. In some embodiments, the amount is in the range of about 1.7 mg, to be administered, 1, 2, 3, 4, 5, 6, 7, or 8 times per day for 1 day or 2 days or for more days if necessary, preferably under a qualified medical practitioner's supervision. The vitamin B₆ can be in the form of pyridoxine HC1. The vitamin B₆ can be delivered by commercially available compositions, such as, for example, Zarbee's Elderberry and honey.

The quantity of the vitamin B₁₂ can vary from about 0.01 μg to about 1000 μg, about 0.1 μg to about 100 μg, or about 1 μg to about 10 μg. In some embodiments, the amount is in the range of about 2.4 μg, to be administered, 1, 2, 3, 4, 5, 6, 7, or 8 times per day for 1 day or 2 days or for more days if necessary, preferably under a qualified medical practitioner's supervision. The vitamin B₁₂ can be in the form of cyanocobalamin. The vitamin B₁₂ can be delivered by commercially available compositions, such as, for example, Zarbee's Elderberry and honey.

In certain embodiments, the treatment can progress through a series of treatment rounds. Each round optimally consists of four treatments that can occur 30 minutes, 1 hour, 2 hours, 4 hours, 8 hours, 10 hours, 12 hours, 14 hours, 16 hours, 24 hours or greater but preferably at about hour 1 to at about hour 4. In preferred embodiments, a Round 1 of dosing treatment is about 4 hours of treatment. A second, different round of dosages (Round 2) can begin approximately 4 to 18 hours after initiation of the first Round 1 of treatment. In certain preferred embodiments, a treatment round is defined as the administration of zinc, zinc ionophores, garlic, vitamins, Echinacea, folate, and/or selenium within one 1-hour period,

In preferred embodiments, Round 1 initiates by the subject drinking about 8 ounces to about a pint of water and then slowly dissolving by mouth a zinc lozenge along with tablets comprising zinc, a zinc ionophore (preferably elderberry), folate, selenium, and vitamins, preferably vitamin C, vitamin D₃, vitamin B₆, vitamin B₁₂ and vitamin E. This dissolving process should take at least 10 minutes, but optimally can extend to 20 minutes, if possible. Then, the subject swallows Echinacea, garlic allicin capsules, and Elderberry capsules. Wait one hour to see if symptoms persist, have increased or have added others, such as a scratchy throat adding to a cough. If, after having taken the first treatment of Round 1, the initial symptomology persists, worsens or leads to additional symptoms, the subject should assume that their health is being threatened by a respiratory virus. They should, therefore, continue with diligence by aggressively following the first treatment of round one with three more identical ones. These treatments are to be spaced out on average in intervals of one to two-hours with no more than three hours between any two treatments. Due to the robustness of the URV2020 procedure, the timing needed for successful treatment can vary over a total time of twelve hours. Thus, if a subject feels symptoms increasing and feels the need to treat themselves aggressively they can treat themselves four times within a four hour period or stretch that timing out to five or more hours, up to a total span of 12 hours. Having completed the four treatments of round one, the subject can assess their symptoms. Having eliminated all concerning symptoms, the subject can stop treatment and assume the virus attack has been defeated. If however symptoms reappear, hang on or worsen, the subject can continue with round two, Preferably, a subject only continues treatment to Round 2 if symptoms reappear.

Round 2 can start within one to two hours of the completion of Round 1's four treatments, but no more than 18 hours from the beginning of Round 1 should lapse before the treatments of Round 2 begin. A need to progress to Round 2 should leave no doubt in a subject's mind that they are under attack by a respiratory virus. Hence, Round 2 should be completed in its entirety of four treatments. Optimally, Round 2 begins 4 to 18 hours after the initiation of the initial hour of Round 1 of treatment. The subject drinks a pint of water, leaving out the zinc lozenge employed in Round 1 treatments to avoid taking too much zinc, dissolves tablets comprising zinc, a zinc ionophore, vitamins, folate, and selenium, preferably vitamin C, vitamin D₃, vitamin B₆, vitamin B₁₂ and vitamin E, and swallows with water garlic, Echinacea, and elderberry. Note that this treatment is the same as the treatments used in Round 1, except that the zinc lozenge is not used in order to keep the subject's total dose of zinc at a safe level that is under 100-mg in a 24 hour period.

Having completed the four treatments of Round 2, if symptoms persist, the subject should restart treatments on the next day, approximately 24 hours after the initiation of Round 1 treatments on the first day. As done on the first day, the treatment schedule begins with the treatments, preferably four treatments, following the exact protocol of Round l′s treatments, preferably four treatments, used on day 1. And, as was true for the first day the timing of the treatments progress based on the presence (or lack thereof) of symptoms. As on the first day, Round 1 treatments taken hourly, preferably over a 4-hour period, should be the minimum time used for treatments during Round 1. Depending on the severity of symptoms, as many as 4 or 5 hours of time between treatments could be employed. Hence, the overall timing for Round 1 in the second day is not so critical that the subject can't adjust the timing based on the intensity of their symptomology. But, since the subject should by now be fairly certain that they are, in fact, dealing with some kind of respiratory virus infection, the subject will want to pay close attention to those symptoms and if those symptoms seem to be increasing in intensity or increasing in numbers the subject should not hesitate to continue being aggressive in their treatment by shortening the time before administering their next treatments. And, if after Round 1 is complete, symptoms persist, the patient should administer Round 2's schedule of treatments, preferably four treatments, just as was done on the first day. Again, each treatment of Round 2 can be administered hourly, preferably over a 4-hour period; however, as already noted, as many as 4 or 5 hours between treatments may be employed if symptoms seem to be abating.

The subject may simply think that he/she has a respiratory virus infection or one or more symptoms consistent with a respiratory virus infection. Some individuals infected with a respiratory virus will not know they have the infection because they will not have symptoms; however, the subject may think that he/she will become sick due to contact with an infected and/or potentially infected person. It is the essence of this invention that it is not necessary to know which of the following virus it is, as listed below. They all should succumb to the methods described herein due to the respiratory location of their invasion site, their RNA polymerase replication system and sensitivity to zinc therein, and their susceptibility via zinc ionophores, and zinc's ability to promote and an individual's first responder immune-support. The following are the virus infections that subjects may not know or suspect they ever had.

The respiratory virus can be an influenza virus. The influenza virus can be influenza A, influenza B, influenza C, or influenza D. Influenza A can be H1N1, H2N2, H3N2, H5N1, H7N7, H1N2, H9N2, H7N2, H7N3, H1ON7, H7N9, or H6N1.

The respiratory virus can be a RSV. The RSV can be RSV-A with genotypes GA1, GA2, GA3, GA4, GA5, GA6, GA7, SAA1, NA1, or NA2 or RSV-B with genotypes GB1, GB2, GB3, GB4, SAB 1, SAB2, SAB3, BA1, BA2, BA3, BA4, BAS, or BA6

The respiratory virus can be a PIV. The PIV can be HPIV-1, HPIV-2, HPIV-3, or HPIV-4.

The respiratory virus can be a hMPV. The hMPV can be from the Al, A2, B1, or B2 lineage.

The respiratory virus can be a rhinovirus, including serotypes A, C, or C.

The respiratory virus can be a coronavirus. In some embodiments of the methods of the invention, the human coronavirus is selected from among SARS-CoV-2, SARS-CoV, and MERS-CoV. SARS-CoV-2 is a novel human coronavirus that causes coronavirus disease 2019, also known as COVID-19 and COVID19. MERS-CoV is the beta coronavirus that causes Middle East Respiratory Syndrome, or MERS. SARS-CoV is the beta coronavirus that causes severe acute respiratory syndrome, or SARS.

In some embodiments of the methods of the invention, the human coronavirus is a common human coronavirus, such as type 229E (an alpha coronavirus), NL63 (an alpha coronavirus), OC43 (a beta coronavirus), and HKU1 (a beta coronavirus).

The symptoms of a respiratory virus infection depend on the type of respiratory virus and severity of the infection. If a subject has a mild to moderate upper-respiratory infection, such as the common cold, symptoms may include: runny nose, headache, cough, sore throat, fever, and general feeling of being unwell. Some respiratory viruses can cause severe symptoms. These infections may turn into bronchitis and pneumonia, which can cause symptoms such as fever (which can be quite high with pneumonia), cough with mucus, shortness of breath, and chest pain or tightness when the subject breaths or coughs.

The clinical spectrum of SARS-CoV-2 may range from mild disease with non-specific signs and symptoms of acute respiratory illness, to severe pneumonia with respiratory failure and septic shock. Asymptomatic infections have also been reported. The upper respiratory nature of this virus is a characteristic that makes the methods and compositions embodied in the subject invention capable of treating the viral infection effectively by inhibiting the further progress of the virus through the appropriate use of zinc, as detailed in our disclosure, which can quickly inactivate viral replication and ultimately kill the virus where it resides in the subjects oral/nasal cavity. The back of a subject's throat is the region that is particularly sensitive. The methods described herein stop the progress of said respiratory virus at their entry point and, thereby, prevent the viral disease progressing to its notorious serious disease symptomologies.

To diagnose respiratory virus infections, healthcare providers typically take a subject's medical history and ask the subject their symptoms, do a physical examination, and may conduct laboratory tests on a biological sample such as blood, or a respiratory specimen such as sputum or a throat swab. However, a major advantage of the methods embodied herein is that the methods are designed to avoid the need of a healthcare-provider's assistance. This avoidance is accomplished by subjects treating themselves and resolving the viral assault before any significant serious symptoms that could otherwise arise from the virus infections have had the chance to develop. Of special note is the urgent need for the present invention, as has been starkly highlighted by the worldwide pandemic of 2019 caused by SARS-CoV-2. To this point, maximizing the benefit of the present invention entails the early recognition by a potentially infected subject of possible viral infection. An example of a subject possibly being infected by SARS-CoV-2 can be particularly illustrative. Positive COVID-19 patients complain of a variety of symptoms, the most common of which include severe tiredness, fever, dry cough, sore throat, loss of taste and smell, headache, diarrhea, and nausea. However, these harsh symptoms are of little value when it comes to the present invention's ability to effectively stop the COVID-19 disease within as little as four hours, but generally within a 24-hour period following the first noticeable signs of viral attack when the treatment process has successfully been activated. The methods described herein are safe and robust enough to allow subjects a free hand at using them at the slightest possible sign suggested by a slight change in each subject's well-being, especially ones identified as new, sudden, ticklish or scratchy sensations in the back of the throat. In other words, without hesitation or concern that the detected changes might not actually be a viral infection, and not wondering if it be a cold, flu, or COVID-19, subjects are to, for example, immediately down a pint of water, preferably lay on their backs, and begin the slow process of dissolving together by mouth a Cold Eeze Zinc Lozenge, comprising 13.3 mg of zinc, and three Zarbee's Elderberry and Honey Total Immune Support Tablets, together comprising 5-mg zinc; 326-mg elderberry fruit blend comprising honey, Elderberry fruit extract, chicory, and baker's yeast beta glucan; 17-mg vitamin B₆; 666-μg folate; 2,4-μg vitamin B₁₇; 300-μg vitamin A, 500-mg vitamin C: 20-μg vitamin D₃; 15-mg vitamin E; and 15-μg selenium. letting the zinc, zinc ionophore, and immune boosting vitamins reach the back of the throats. When the lozenge and tablets are completely dissolved, a process that should take about 10 to about 15 minutes, the subjects swallow, for example, with water or any other liquid, a New Chapter Elderberry Capsule comprising 335-mg of European Elderberry and 50 mg Black Currant “true-to-fruit” membrane extraction, two 400 mg Nature's Bounty Echinacea Capsules, and two Nature's Bounty Garlic tablets, each tablet comprising 2000 mg of garlic. Subjects then wait for one to two hours to assess their condition. Due to the robustness of the method, the initial time taken for this assessment is not critical, but no more than two hours should lapse before a decision is made. If a subject no longer feels the initial or any possible slight new symptoms, there is no need to continue the treatment, unless the subject wants to take one more treatment as a precautionary treatment. If, however, the symptomology of scratchy throat, temperature wobble, or sickish feeling remains or has gotten worse, say, for example, with the addition of chills, slight fever, dry cough, headache or a worse scratchy throat, a subject should move on to repeat Round 1 of the therapy with three more identical administrations of the dosages taken initially. This is done every hour or two for the three times just mentioned and with each treatment preceded by a pint of water drunk just before taking the Cold Eeze lozenge and Zarbee's dissolvable tablets, and not to forget it should be done while the subject reclines to better coat the back of the throat with the zinc treatment.

Having treated the infection with success, it may be of great interest to identify the invading virus. For example, it has become especially critical to know if a potentially infected person has indeed contracted the virus responsible for the COVID-19 disease. In some embodiments, a molecular assay may be used to detect the presence or absence of a respiratory virus in a biological sample from the subject. For example, several assays that detect SARS-CoV-2 have been under development. Some assays may detect only the novel virus and some may also detect other strains (e.g., SARS-CoV) that are genetically similar. The table below (Table 2) is a summary of some available protocols and their gene targets.

TABLE 2

| Country | Institution | Gene targets |
|---|---|---|
| United States | US CDC | Three N primers, |
| | | RdRP |
| China | China CDC | ORF1ab and N |
| Germany | Charité | RdRP, E, N |
| Hong Kong | HKU | ORF1b-nsp14, N |
| Japan | National Institute of | Pancorona and |
| | Infection Diseases, | multiple targets, Spike |
| | Department of | protein |
| | Virology III | |
| Thailand | National Institute of | N |
| | Health | |

China CDC Primers and probes for detection 2019-nCoV (24 Jan. 2020)
Diagnostic detection of Wuhan coronavirus 2019 by real-time RT-PCR-Charité, Berlin Germany (17 Jan. 2020)
Detection of 2019 novel coronavirus (2019-nCoV) in suspected human cases by RT-PCR-Hong Kong University (23 Jan. 2020)
PCR and sequencing protocol for 2019-nCoV-Department of Medical Sciences, Ministry of Public Health, Thailand (Updated 28 Jan. 2020)
PCR and sequencing protocols for 2019-nCoV-National Institute of Infectious Diseases Japan (24 Jan. 2020)
US CDC Real-Time RT-PCR Panel for Detection 2019-Novel Coronavirus (28 Jan. 2020)
US CDC panel primer and probes-U.S. CDC, USA (28 Jan. 2020)
(“WHO interim guidance for laboratory testing for 2019 novel coronavirus (2019-nCoV) in humans” from World Health Organization website).

SARS-CoV-2 RNA has been detected from upper and lower respiratory tract specimens, and the virus has been isolated from upper respiratory tract specimens and bronchoalveolar lavage fluid. SARS-CoV-2 RNA has been detected in blood and stool specimens. The duration of SARS-CoV-2 RNA detection in the upper and lower respiratory tracts and in extrapulmonary specimens has not been determined. It is possible that RNA could be detected for weeks, which has occurred in some cases of MERS-CoV or SARS-CoV infection. Viable SARS-CoV has been isolated from respiratory, blood, urine, and stool specimens, and viable MERS-CoV has been isolated from respiratory tract specimens.

Subjects may be in contact with infected individuals. In certain embodiments, a physician may advise a subject to use the present inventive methods for remediation and convalescence. Treatment methods optionally include steps of advising that the subject get plenty of rest and drink fluids for hydration and administration of agents that alleviate symptoms of a respiratory virus infection, such as those that reduce fever and pain (e.g., acetaminophen and/or paracetamol), particularly for common respiratory virus infections. The methods may include administration of the fluids to the subject for hydration. In certain embodiments, at least 0.5, 1, 1.5, 2, 2.5, 3, 3.5, or 4 pints of water or greater amounts of water can be administered before, after, or during the administration of zinc, zinc ionophores, garlic, vitamins, Echinacea, folate, and/or selenium. The administration of water can transport viruses to the stomach. In preferred embodiments, the water is consumed rapidly, i.e., as quickly as possible.

The subject may be any age or gender. In some cases, the subject may be an infant or older adult. In some embodiments, the subject is 40 years of age or older. In some embodiments, the subject is 55 years of age or older. In some embodiments, the subject is 60 years of age or older. In some embodiments, the subject (of any age or gender) has heart or lung disease, diabetes, or a weakened immune system.

In certain embodiments, the administration of zinc, zinc ionophores, garlic, vitamins, Echinacea, folate, and/or selenium occurs within 3, 6, 12, 18, 24, 36, 48, 60, 72, 84, or 96 hours of the onset of viral infection symptoms that can include fever, chills, coughing, runny nose, sneezing, loss of smell, loss of appetite, muscle pain, and/or joint pain. Furthermore, a subject can begin the administration of zinc, zinc ionophores, garlic, vitamins, Echinacea, folate, and/or selenium before the onset of any symptoms, preferably if the subject has been in contact or shared a space with an individual with either viral infection symptoms or a confirmed viral infection. In certain embodiments, when the administration of zinc, zinc ionophores, garlic, vitamins, Echinacea, folate, and/or selenium is administered within at least 24 hours, 36 hours, 48 hours, 60 hours, 72 hours, 84 hours, 96 hours, or greater, repeated administrations of zinc, zinc ionophores, garlic, vitamins, Echinacea, folate, and/or selenium can be used to treat a subject in need.

The invention further provides kits including zinc, zinc ionophore, garlic, folate, selenium, Echinacea, and/or vitamins and pharmaceutical formulations, packaged into suitable packaging material, optionally in combination with instructions for using the kit components, e.g., instructions for performing a method of the invention. In one embodiment, a kit includes an amount of zinc, zinc ionophore, garlic, folate, selenium, Echinacea, and/or vitamins, and instructions for administering the inhibitor compositions to a subject in need of treatment on a label or packaging insert. In further embodiments, a kit includes an article of manufacture, for delivering the inhibitor compositions into a subject locally, regionally or systemically, for example.

As used herein, the term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention, e.g., treating a respiratory virus infection, an assay for identifying a subject having a respiratory virus infection, etc. Thus, in additional embodiments, a kit includes a label or packaging insert including instructions for practicing a method of the invention in solution, in vitro, in vivo, or ex vivo.

Instructions can therefore include instructions for practicing any of the methods of the invention described herein. For example, pharmaceutical compositions can be included in a container, pack, or dispenser together with instructions for administration to a subject to treat a respiratory virus infection. Instructions may additionally include appropriate administration route, dosage information, indications of a satisfactory clinical endpoint or any adverse symptoms that may occur, storage information, expiration date, or any information required by regulatory agencies such as the Food and Drug Administration or European Medicines Agency for use in a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Kits can additionally include a buffering agent, a preservative, or an agent for stabilizing the zinc, zinc ionophore, garlic, folate, selenium, Echinacea, and/or vitamins. The kit can also include control components for assaying for the presence of a respiratory virus, e.g., a control sample or a standard. Each component of the kit can be enclosed within an individual container or in a mixture and all of the various containers can be within single or multiple packages. In certain embodiments, the subject methods and kits can be used to identify and/or treat asymptomatic individuals that are infected with SARS-CoV-2. To treat asymptomatic individuals, they can be provided with kits that possess the components and instructions to perform the methods of the subject invention, preferably the URV2020 method, hereinafter referred to as the URV2020 kit. The URV2020 kits can also be provided to asymptomatic individuals that are not infected by SARS-CoV-2.

In certain embodiments, to identify and/or treat asymptomatic individuals and/or determine the efficacy of the methods of the subject invention, a double blind test can be performed. Numerically labelled kits can be provided to asymptomatic individuals; the asymptomatic individuals are preferably infected with SARS-CoV-2. About 50% of the kits can possess the ingredients and instructions to perform the methods of the subject invention, preferably the URV2020 method (URV2020 kit). About 50% of the kits provided can be a placebo, in which the means to perform the methods of the subject invention are not provided. The use of an approximately equal number of placebo and URV2020 kits would automatically divide the test subjects into two large, randomly selected groups of individuals. The individuals can be tested for SARS-CoV-2 infection just prior to receipt of a kit. The individuals can be tested hourly, daily, every other day, and/or weekly for SARS-CoV-2 until all individuals have tested negative for SARS-CoV-2. In order to determine the efficacy of the methods of the subject invention, preferably the URV2020 method and corresponding URV2020 kit, the length of time between the test immediately preceding receipt of the kit and a negative test can be determined. If individuals receiving the URV2020 kit are testing negative for SARS-CoV-2 sooner than the placebo group on a significant, statistical average, the methods of the subject invention will be determine to be effective.

### Definitions

As used herein, a subject is “in need of” a treatment if such human subject would benefit biologically, medically, or in quality of life from such treatment (preferably, a human). In some embodiments, the subject has a respiratory virus infection and is in need of therapy. In other embodiments, the subject does not have a respiratory virus infection and is in need of prophylaxis. In some embodiments, the subject in need of prophylaxis is at risk of becoming infected with a respiratory virus. In some embodiments, the subject is at increased risk of becoming infected with a respiratory virus relative to others in the population.

As used herein, the terms “subject”, “patient”, and “individual” refer to a human of any age or gender.

As used herein, the term “treat”, “treating”, or “treatment” of any disease or disorder refers, in one embodiment, to ameliorating the disease or disorder (i.e., slowing or arresting or reducing the development of the disease or at least one of the clinical symptoms thereof). In another embodiment “treat”, “treating”, or “treatment” refers to alleviating or ameliorating at least one physical parameter including those which may not be discernible by the subject. In yet another embodiment, “treat”, “treating”, or “treatment” refers to modulating the disease or disorder, either physically, (e.g., stabilization of a discernible symptom), physiologically, (e.g., stabilization of a physical parameter), or both. In yet another embodiment, “treat”, “treating”, or “treatment” refers to prophylaxis (preventing or delaying the onset or development or progression of the disease or disorder).

As used herein, the term “administration” is intended to include, but is not limited to, the following delivery methods: topical, oral, parenteral, subcutaneous, transdermal, transbuccal, intravascular (e.g., intravenous or intra-arterial), intramuscular, subcutaneous, intranasal, and intra-ocular administration. Administration can be local at a particular anatomical site, such as a site of infection, or systemic.

As used herein, the term “contacting” in the context of contacting a cell with at least zinc composition in vitro or in vivo means bringing at least one inhibitor into contact with the cell, or vice-versa, or any other manner of causing the inhibitor and the cell to come into contact.

As used herein, the phrase “upper respiratory viral infection” or “URV infection” is a viral infection that has a primary mode of transmission by droplet, airborne particle, or other aerosolized form. The droplet(s) containing the viral particles initiates infection by contacting the upper respiratory tract, including nose, nasal passage, paranasal sinuses, the pharynx, larynx, vocal chords, tongue, tracheae, hard palate, nostril, oral cavity, and/or gums. An upper respiratory viral infection can infect additional areas of the body including the lower respiratory tract, blood vessels, kidney, and or liver.

As used herein, the term “interact” in the context of viral transmission between humans refers to physical contact including touching a person or surface, hand shaking, patting, hugging, and kissing; and non-physical contact that has been demonstrated to transmit viruses including, movement of aerosolized viral particles including coughing, breathing, laughing, sneezing, singing, talking, toilet flushing, and yelling; and airborne transmission by which an infected individual can leave viral particles in the air and an uninfected individual can contact the viral particle without even being in the same room at the same time as the infected individual.

The compounds of the present invention can be formulated into pharmaceutically-acceptable salt forms. Pharmaceutically-acceptable salts of the compounds of the invention can be prepared using conventional techniques. “Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts described herein are pharmaceutically acceptable acid addition salts and pharmaceutically acceptable base addition salts.

“Pharmaceutically acceptable acid addition salt” refers to those salts that retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, a “derivative” or “pharmaceutically active derivative” refers to any compound that upon administration to the recipient, is capable of providing directly or indirectly, the activity disclosed herein (e.g., anti-respiratory virus activity). The term “indirectly” also encompasses “prodrugs” which may be converted to the active form of the drug, e.g., via endogenous enzymes or metabolism (biotransformation). The prodrug is a derivative of the compounds according to the invention and presenting anti-respiratory virus activity that has a chemically or metabolically decomposable group, and a compound that may be converted into a pharmaceutically active compound according to the invention in vivo by solvolysis under physiological conditions. The prodrug is converted into a compound according to the present invention by a reaction with an enzyme, gastric acid or the like under a physiological condition in the living body, e.g., by oxidation, reduction, hydrolysis or the like, each of which is carried out enzymatically. These compounds can be produced from compounds of the present invention according to well-known methods. The term “indirectly” also encompasses metabolites of compounds according to the invention. Chemical reactions, reactants, and reagents useful for making derivatives can be found, for example, in March's Advanced Organic Chemistry, 7^{th} edition, 2013, Michael B. Smith, which is incorporated herein by reference in its entirety.

More specifically, the term “prodrug” refers to a chemical compound that can be converted by the body (i.e., biotransformed) to another chemical compound that has pharmacological activity. The prodrug may itself have pharmacological activity before conversion, or be inactive before conversion and activated upon conversion. Active prodrugs or inactive prodrugs of compounds of the invention may be administered to a subject or contacted with a cell in vitro or in vivo. Instead of administering a drug directly, a prodrug may be used instead to improve how a drug is absorbed, distributed, metabolized, and excreted (ADME). For example, a prodrug may be used to improve bioavailability when a drug itself is poorly absorbed from the gastrointestinal tract, or to improve how selectively the drug interacts with cells or processes that are not its intended target, which can reduce adverse or unintended effects of a drug. Major types of prodrugs include, but are not limited to, type I prodrugs, which are biotransformed inside cells (intracellularly), and type II prodrugs, which are biotransformed outside cells (extracellularly), such as in digestive fluids or in the body's circulatory system. These types can be further categorized into subtypes based on factors such as whether the intracellular bioactivation location is also a site of therapeutic action, or whether or not bioactivation occurs in the gastrointestinal fluids or in the circulation system (Wu, Kuei-Meng, “A New Classification of Prodrugs: Regulatory Perspectives, Pharmaceuticals, 2009, 2(3):77-81, which is incorporated by reference herein in its entirety)”.

The term “metabolite” refers to all molecules derived from any of the compounds according to the present invention in a cell or organism, preferably mammal. Pharmaceutically active metabolites of the compounds of the invention may be administered to a subject or contacted with a cell in vitro or in vivo.

The phrase “pharmaceutically acceptable” indicates that the substance or composition must be compatible chemically and/or toxicologically, with the other ingredients comprising a formulation, and/or the mammal being treated therewith.

Pharmaceutical formulations include “pharmaceutically acceptable” and “physiologically acceptable” carriers, diluents or excipients. In this context, the terms “pharmaceutically acceptable” and “physiologically acceptable” include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral and antifungal agents) can also be incorporated into the compositions.

The phrase “effective amount” means an amount of an agent, such as a zinc composition, that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

As used herein, a subject is “in need of” a treatment if such human or non-human animal subject would benefit biologically, medically or in quality of life from such treatment (preferably, a human).

As used herein, the term “inhibit”, “inhibition”, or “inhibiting” refers to the reduction or suppression of a given condition, symptom, or disorder, or disease (e.g., respiratory virus infection, or respiratory viral load or titer), or a significant decrease in the baseline activity of a biological activity or process.

As used herein, the phrases “stopping a virus” or “stopping a respiratory virus infection” refer to the process of disrupting the progress of a respiratory virus's replication and disease production by early detection and treatment of faint symptoms prior to said virus having a chance to create the easily recognizable, debilitating symptoms that characterize a cold, flu, coronavirus, or other respiratory virus.

The terms “compounds of the present invention” or “agents of the invention” (unless specifically identified otherwise) refer to zinc, zinc ionophore, garlic, folate, selenium, Echinacea, and/or vitamins including salts thereof, as well as all stereoisomers (including diastereoisomers and enantiomers), rotamers, tautomers, and isotopically labeled compounds (including deuterium substitutions), as well as inherently formed moieties (e.g., polymorphs, solvates and/or hydrates). For purposes of this invention, solvates and hydrates are generally considered compositions.

The terms “a,” “an,” “the,” and similar terms used in the context of the present invention (especially in the context of the claims) are to be construed to cover both the singular and plural unless otherwise indicated herein or clearly contradicted by the context. For example, the term “cell” includes a singular cell and a plurality of cells unless specified to the contrary; and the term “inhibitor” includes a singular inhibitor and a plurality of inhibitors.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### EXAMPLES

### Example 1

### The URV2020 Method

Round 1 of treatment commences immediately after a subject has identified any new, slightly uncomfortable feeling or irritating change. Examples of such changes include minor throat irritations, body temperature fluctuations or sickish feelings. In the first treatment of Round 1 subjects first rapidly drink a pint of water. Next, they slowly dissolve by mouth a lozenge comprising 13.3 mg zinc, such as that found in a Cold Eeze lozenge, along with three tablets that comprise a totals of 5-mg zinc; 326-mg elderberry fruit blend comprising honey, Elderberry fruit extract, chicory, and baker's yeast beta glucan; 1.7-mg vitamin B₆; 666-μg folate; 2.4-μg vitamin B₁₂; 300-μg vitamin A, 500-mg vitamin C; 20-μg vitamin D₃; 15-mg vitamin E; and 15-μg selenium, such as that found in three Zarbee's Elderberry and Honey Chewable Tabs. The dissolving process should take at least ten minutes or longer. Zinc helps activate immunity system first-responders such as killer T-cells and cytokines, and zinc can interfere with viral RNA-dependent, RNA polymerase. The Elderberry additive has been shown to work as an effective zinc ionophore, which can help zinc enter infected cells to inactivate the virus as it attempts to replicate in the subject's throat. The vitamins and selenium can to enhance the immune system by stimulating antibody-producing lymphocytes.

After the tablets and lozenge have dissolved, the subject swallows in one capsule 335-mg of European Elderberry and 50 mg Black Currant “true-to-fruit” membrane extraction, such as found in one capsule of NewChapter Elderberry Force, 800 mg of Echinacea, such as found in two capsules of Nature's Bounty Whole Herb Echinacea, and 4000 mg of Garlic, such as found in two capsules of Nature's Bounty Heart Health Garlic.

Example 2

### Second Embodiment of Treatment of a Respiratory Virus Infection

The subject can use a table to keep track of the symptoms. To use the table, use body temperature for Fever, otherwise use Y for Yes and blank for No. The chart starts at Hour One of suspected disease onset. The table is designed to record results for each of the following 23 hours. New forms can be used if treatments take place beyond 24 hours. Table 3 provides an example of the symptoms of Patient A that respond to the treatment and resolve within 24 hours; therefore, in this embodiment the patient would not need to continue treatments on day 2. Under the “Items used” section of Table 3, 1 stands for the administration of water; 2 for 13.3 mg zinc in the form of a lozenge (e.g., Cold Eeze); 3 for three tablets taken together with the lozenge and comprising 5-mg of zinc, 326-mg blend of elderberry, 1.7-mg vitamin B₆, 666-μg folate, vitamin B₁₂, 300-μg vitamin A, 500-mg vitamin C, 20-μg vitamin D₃, 15-mg vitamin E, and 15-μg selenium (e.g., Zarbee's Elderberry and honey); 4 is 335-mg Elderberry (e.g., one New Chapter Elderberry capsule); 5 is 4000-mg Garlic allicin (e.g, two 2000 mg Nature's Bounty garlic allicin capsule); and 6 is 800-mg Echinacea (e.g., two 400-mg Nature's Bounty Echinacea capsules).

At 10:00 AM, Patient A has a scratchy throat, a fever, and stuffy nose, so Patient A initiates a Round 1 treatment in which the patient administers a pint of water by rapidly drinking the water, a 13.3-mg zinc lozenge and 3 tablets together comprising 5-mg of zinc; 326-mg elderberry fruit blend comprising honey, elderberry fruit extract, chicory, and baker's yeast beta glucan; 1.7-mg vitamin B₆; 666-μg folate; 2.4-μg vitamin B₁₂; 300-μg vitamin A, 500-mg vitamin C; 20-μg vitamin D₃; 15-mg vitamin E; and 15-μg selenium by dissolving in the mouth. Then, Patient A swallows a 335-mg Elderberry fruit extract capsule, two 2000-mg garlic allicin capsules, and two 400-mg Echinacea capsules.

At 11:00 AM, Patient A has a scratchy throat, cough, congestion, a fever, stuffy nose, and an achy feeling, so Patient A repeats the Round I treatment in which the patient administers a pint of water by rapidly drinking the water, a 13.3-mg zinc lozenge and 3 tablets together comprising 5-mg of zinc; 326-mg elderberry fruit blend comprising honey, elderberry fruit extract, chicory, and baker's yeast beta glucan; 1.7-mg vitamin B₆; 666-μg folate; 2.4-μg vitamin B₁₂; 300-μg vitamin A, 500-mg vitamin C; 20-μg vitamin D₃; 15-mg vitamin E; and 15-μg selenium by dissolving in the mouth. Then, Patient A swallows a 335-mg Elderberry fruit extract capsule, two 2000-mg garlic allicin capsules, and two 400-mg Echinacea capsules.

At 12:00 PM, Patient A has a scratchy throat, congestion, and a stuffy nose. Patient A initiates a Round 2 treatment in which the patient administers a pint of water by rapidly drinking the water and a 13.3 mg zinc lozenge by dissolving in the mouth. Then, Patient A swallows a 335 mg Elderberry fruit extract capsule, two 2000 mg garlic allicin capsules, and two 400 mg Echinacea capsules.

At 1:00 PM, Patient A has a stuffy nose, Patient A repeats a Round 1 treatment in which the patient administers a pint of water by rapidly drinking the water, a 13.3 mg zinc lozenge and 3 tablets together comprising 5 mg of zinc; 326 mg elderberry fruit blend comprising honey, elderberry fruit extract, chicory, and baker's yeast beta glucan; 1.7 mg vitamin B₆; 666 μg folate; 2,4 μg vitamin B₁₂; 300 μg vitamin A, 500 mg vitamin C; 20 μg vitamin D₃; 15 mg vitamin E; and 15 μg selenium by dissolving in the mouth. Then, Patient A swallows a 335 mg Elderberry fruit extract capsule, two 2000 mg garlic allicin capsules, and two 400 mg Echinacea capsules.

At 2:00 PM, Patient A has no symptoms. Patient A repeats a Round 2 treatment in which the patient administers a pint of water by rapidly drinking the water and a 13.3 mg zinc lozenge by dissolving in the mouth. Then, Patient A swallows a 335 mg Elderberry fruit extract capsule, two 2000 mg garlic allicin capsules, and two 400 mg Echinacea capsules.

At 7:00 PM, Patient A has a stuffy nose, but does not administer a treatment.

At 11:00 PM, Patient A has a stuffy nose. Patient A repeats a Round 1 treatment in which the patient administers a pint of water by rapidly drinking the water, a 13,3 mg zinc lozenge and 3 tablets together comprising 5 mg of zinc; 326 mg elderberry fruit blend comprising honey, elderberry fruit extract, chicory, and baker's yeast beta glucan; 1.7 mg vitamin B₆; 666 μg folate; 2.4 μg vitamin B₁₂; 300 mg vitamin A, 500 mg vitamin C; 20 μg vitamin D₃; 15 mg vitamin E; and 15 μg selenium by dissolving in the mouth. Then, Patient A swallows a 335 mg Elderberry fruit extract capsule, two 2000 mg garlic allicin capsules, and two 400 mg Echinacea capsules.

At 1:00 AM of the following day, Patient A has no symptoms. Patient A repeats the Round 2 treatment in which the patient administers a pint of water by rapidly drinking the water and a 13.3 mg, zinc lozenge by dissolving in the mouth. Then, Patient A swallows a 335 mg Elderberry fruit extract capsule, two 2000 mg garlic allicin capsules, and two 400 mg Echinacea capsules.

At 2:00 AM, Patient A has no symptoms. Patient A administers a pint of water,

At 4:00 AM, Patient A has a stuffy nose. Patient A administers a pint of water.

At 6:00 AM, Patient A has no symptoms. Patient A administers a pint of water. The Patient has no symptoms after 24 hours, so Patient A does not continue treatment, The following is an early symptom data record table that was used by the inventor and recorded as an early experience in treating a putative RNA respiratory virus infection with the URV2020 method.

TABLE 3

| Hr, Date | | Scratchy | Cough/ | Fever/ | Stuffy | Unusual | Achy | | ? or P | Items |
|---|---|---|---|---|---|---|---|---|---|---|
| & Time | | Throat | Congestion | Chills | Nose | Pain | Feeling | Nausea | Precautionary | used |
| Jan. 18, 2020 | | Y | | Y | Y | | | | ? | 123456 |
| 1 | 10 am | | | | | | | | | |
| 2 | 11 am | Y | Y both | Y | Y | | Y | | | 123456 |
| 3 | 12 pm | Y | Congestion | | Y | | | | | 12 456 |
| | | | only | | | | | | | |
| 4 | 1 pm | | | | Y | | | | | 123456 |
| 5 | 2 pm | | | | | | | | | 12 456 |
| 6 | 4 pm | | | | | | | | | |
| 7 | 5 pm | | | | | | | | | |
| 8 | 6 pm | | | | | | | | | |
| 9 | 7 pm | | | | Y | | | | | |
| 10 | 8 pm | | | | | | | | | |
| 11 | 9 pm | | | | | | | | | |
| 12 | 10 pm | | | | | | | | | |
| 13 | 11 pm | | | | Y | | | | P | 123456 |
| 14 | 12 pm | | | | | | | | | |
| 15 | 1 am | | | | | | | | P | 12 456 |
| 16 | 2 am | | | | | | | | | 1 |
| 17 | 3 am | | | | | | | | | |
| 18 | 4 am | | | | Y | | | | P | 1 |
| 19 | 5 am | | | | | | | | | |
| 20 | 6 am | | | | | | | | P | 1 |
| 21 | 7 am | | | | | | | | | |
| 22 | 8 am | | | | | | | | | |
| 23 | 9 am | | | | | | | | | |
| 24 | 10 am | | | | | | | | | |

The following are two additional informational-only examples that could have ensued based on many conversations the inventor has had with users of the URV2020 methodology.

### Example 3

### A Third Embodiment of the URV2020 Method

Round 1 of treatment commences immediately after a subject has identified any new, slightly uncomfortable feeling or irritating change. Examples of such changes include minor throat irritations, body temperature fluctuations or sickish feelings. In the first treatment of Round 1 subjects first rapidly drink 8 to 16 ounces of water, and with the water swallow 335-mg of European Elderberry and 50 mg Black Currant “true-to-fruit” membrane extraction, such as found in one capsule of NewChapter Elderberry Force, 800 mg of Echinacea, such as found in two capsules of Nature's Bounty Whole Herb Echinacea, and 4000 mg of Garlic, such as found in two capsules of Nature's Bounty Heart Health Garlic.

Next, they slowly dissolve by mouth a lozenge comprising 13.3 mg zinc, such as that found in a Cold Eeze lozenge, along with three tablets that comprise a totals of 5-mg zinc; 326-mg elderberry fruit blend comprising honey, Elderberry fruit extract, chicory, and baker's yeast beta glucan; 1.7-mg vitamin B₆; 666-μg folate; 2.4-μg vitamin B₁₂; 300μg vitamin A, 500-mg vitamin C; 20μg vitamin D₃; 15-mg vitamin E; and 15-μg selenium, such as that found in three Zarbee's Elderberry and Honey Chewable Tabs. The dissolving process should take at least ten minutes or longer. Zinc helps activate immunity system first-responders such as killer T-cells and cytokines, and zinc can interfere with viral RNA-dependent, RNA polymerase. The Elderberry additive has been shown to work as an effective zinc ionophore, which can help zinc enter infected cells to inactivate the virus as it attempts to replicate in the subject's throat. The vitamins and selenium can to enhance the immune system by stimulating antibody-producing lymphocytes.

Subjects can then initiate a Round 2 dosing treatment in which each subject administers 8-16 ounces of water by rapidly drinking the water, and with the water they swallow a 335 mg Elderberry fruit extract capsule, two 2000 mg garlic allicin capsules, and two 400 mg Echinacea capsules. Then, the subjects administer 3 tablets together comprising 5 mg of zinc; 326 mg, elderberry fruit blend comprising honey, elderberry fruit extract, chicory, and baker's yeast beta glucan 1.7 mg vitamin B₆; 666 μg folate; 2.4 vitamin B₁₂; 300 μg vitamin A, 500 mg vitamin C; 20 μg vitamin D₃; 15 mg vitamin E; and 15 μg selenium by dissolving in the mouth.

### Example 4

### The Use of Round 1 and Round 2 of URV2020 Method

At 10:00 AM, Patient B has a scratchy throat, a fever, and stuffy nose, so Patient B initiates a Round 1 dosing treatment in which the patient administers a pint of water by rapidly drinking the water, a 13.3 mg zinc lozenge and 3 tablets together comprising 5 mg of zinc; 326 mg elderberry fruit blend comprising honey, elderberry fruit extract, chicory, and baker's yeast beta glucan; 1.7 mg vitamin B₆; 666 μg foliate; 2.4 μg vitamin B₁₂; 300 μg vitamin A, 500 mg vitamin C; 20 μg vitamin D₃; 15 mg vitamin E; and 15 μg selenium by dissolving in the mouth. Then, Patient B swallows a 335 mg Elderberry fruit extract capsule, two 2000 mg garlic allicin capsules, and two 400 mg Echinacea capsules.

At 11:00 AM, Patient B has a scratchy throat, cough, congestion, a fever, stuffy nose, and an achy feeling. Patient 13 repeats the found 1 treatment in which the patient administers a pint of water by rapidly drinking the water, a 13.3 rng zinc lozenge and 3 tablets together comprising 5 mg of zinc; 326 mg elderberry fruit blend comprising honey, elderberry fruit extract, chicory, and baker's yeast beta glucan; 1.7 mg vitamin B₆; 666 μg folate; 2.4 μg vitamin B₁₂; 300 μg vitamin A, 500 mg vitamin C; 20 μg vitamin D₃; 15 mg vitamin E; and 15 μg selenium by dissolving in the mouth. Then, Patient B swallows a 335 mg Elderberry fruit extract capsule, two 2000 mg garlic allicin capsules, and two 400 mg Echinacea capsules.

At 12:00 PM, Patient B has a scratchy throat, congestion, and a stuffy nose. Patient B repeats the Round 1 treatment in which the patient administers a pint of water by rapidly^{,} drinking the water, a 13.3 mg zinc lozenge and 3 tablets together comprising 5 mg of zinc; 326 mg elderberry fruit blend comprising honey, elderberry fruit extract, chicory, and baker's yeast beta glucan; 1,7 mg vitamin B₆; 666 μg folate; 2.4 μg vitamin B₁₂; 300 μg vitamin A, 500 mg vitamin C; 20 μg vitamin D₃; 15 mg vitamin E; and 15 μg selenium by dissolving in the mouth. Then, Patient B swallows a 335 mg Elderberry fruit extract capsule, two 2000 mg garlic allicin capsules, and two 400 mg Echinacea capsules.

At 1:00 PM, Patient B has a scratchy throat, congestion, and a stuffy nose. Patient B repeats the found 1 treatment in which the patient administers a pint of water by rapidly drinking the water, a 13.3 mg zinc lozenge and 3 tablets together comprising 5 mg of zinc; 326 mg elderberry fruit blend comprising honey, elderberry fruit extract, chicory, and baker's yeast beta glucan; 1.7 mg vitamin B₆; 666 μg folate; 2.4 μg vitamin B₁₂; 300 μg vitamin A, 500 mg vitamin C; 20 μg vitamin D₃; 15 mg vitamin E; and 15 μg selenium by dissolving in the mouth. Then, Patient B swallows a 335 mg Elderberry fruit extract capsule, two 2000 mg garlic allicin capsules, and two 400 mg Echinacea capsules. Patient B then temporarily suspends treatment after having administered four Round 1 dosings.

At 10:00 PM, Patient B has a stuffy nose. Patient B initiates a Round 2 dosing treatment in which the patient administers a pint of water by rapidly drinking the water and 3 tablets together comprising 5 mg of zinc; 326 mg elderberry fruit blend comprising honey, elderberry fruit extract, chicory, and baker's yeast beta glucan; 1.7 mg vitamin B₆; 666 pg folate; 2.4 pg vitamin B₁₂; 300 μg vitamin A, 500 mg vitamin C; 20 μg vitamin D₃; 15 mg vitamin E; and 15 μg selenium by dissolving in the mouth. Then, Patient B swallows a 335 mg Elderberry fruit extract capsule, two 2000 mg garlic allicin capsules, and two 400 mg Echinacea capsules.

At 11:00 PM, Patient B has a stuffy nose, Patient B repeats the Round 2 treatment in which the patient administers a pint of water by rapidly drinking the water and 3 tablets together comprising 5 mg of zinc; 326 mg elderberry fruit blend comprising honey, elderberry fruit extract, chicory, and baker's yeast beta glucan; 1.7 mg vitamin B₆; 666 μg folate; 2.4 μg vitamin B₁₂; 300 μg vitamin A, 500 mg vitamin C; 20 μg vitamin D₃; 15 mg vitamin E; and 15 μg selenium by dissolving in the mouth, Then, Patient B swallows a 335 mg Elderberry fruit extract capsule, two 2000 mg garlic allicin capsules, and two 400 mg Echinacea capsules.

At 12:00 AM of the following day, Patient B has a stuffy nose. Patient B repeats the Round 2 treatment in which the patient administers a pint of water by rapidly drinking the water and 3 tablets together comprising 5 mg of zinc; 326 mg elderberry fruit blend comprising honey, elderberry fruit extract, chicory, and baker's yeast beta glucan; 1.7 mg vitamin B₆; 666 μg folate; 2.4 μg vitamin B₁₂; 300 μg vitamin A, 500 mg vitamin C; 20 μg vitamin D₃; 15 mg vitamin E; and 15 μg selenium by dissolving in the mouth. Then, Patient B swallows a 335 mg Elderberry fruit extract capsule, two 2000 mg garlic allicin capsules, and two 400 mg Echinacea capsules.

At 1:00 AM, Patient B has a runny nose. Patient B repeats the Round 2 treatment in which the patient administers a pint of water by rapidly drinking the water and 3 tablets together comprising 5 mg of zinc; 326 mg elderberry fruit blend comprising honey, elderberry fruit extract, chicory, and baker's yeast beta glucan; 1.7 mg vitamin B₆; 666 μg folate; 2.4 μg vitamin B₁₂; 300 μg vitamin A, 500 mg vitamin C; 20 μg vitamin D₃; 15 mg vitamin E; and 15 μg selenium by dissolving in the mouth. Then, Patient B swallows a 335 mg Elderberry fruit extract capsule, two 2000 mg garlic allicin capsules, and two 400 mg Echinacea capsules. Patient B temporarily suspends treatment after having administered four Round 2 dosings.

At 6:00 AM, Patient B has no symptoms, so Patient B administers a pint of water. The patient has no symptoms after 24 hours, so Patient B does not continue treatment.

### Example 5

### Prophylactic Viral Infection Method of Treatment

At 10:00 AM, Patient D does not have any symptoms; however Patient D spent the previous day inside an office building with a coworker who was coughing and complained of a lack of tasting his breakfast. Patient D initiates Round 1 treatment in which the patient administers a pint of water by rapidly drinking the water, a 13.3 mg zinc lozenge and 3 tablets together comprising 5 mg of zinc; 326 mg elderberry fruit blend comprising honey, elderberry fruit extract, chicory, and baker's yeast beta glucan; 1.7 mg vitamin B₆; 666 μg folate; 2.4 μg vitamin B₁₂; 300 μg; vitamin A, 500 mg vitamin C; 20 μg vitamin D₃; 15 mg vitamin E; and 15 μg selenium by dissolving in the mouth. Then, Patient D swallows a 335 mg Elderberry fruit extract capsule, two 2000 mg garlic allicin capsules, and two 400 mg Echinacea capsules.

At 11:00 AM, Patient D has a no discernible symptoms, so Patient D concludes treatment.

### Example 6 Using the Training Data Record to Record Treatment of Respiratory Virus Infection

Table 4 illustrates an example of a Record of symptoms that were successfully treated and followed by a precautionary treatment. Table 5 illustrates a false alarm that, just to be sure, was also followed by a precautionary treatment.

TABLE 4
Example 1 - Hour 1, military time 06:15. URV attack confirmed at 09:30
is resolved by 19:00 with precautionary treatment at 21:00
URV2020 24-Hr Symptom-Data-Trainer
Date _ _: _ _ : _ _ _ _, Time of onset _ _: _ _ am/pm (circle one)
Zip Code _____; Age___: coded ID ____: Estimate health prior to onset ________.
Use ~° F. for Fever, Y for Yes, and a Blank for No. The chart starts at Hour One of
suspected disease onset. Record results for each of the following 23 hours after that.
Use new forms for any reports after hour 24. Share below known cold, flu, or
COVID19 contacts + any events not included in the chart.

| Hour + | | | | | | | | | Fever | | |
|---|---|---|---|---|---|---|---|---|---|---|---|
| military | | S | T | C | S | S | Sore | | in | | Remedy |
| Time | | T | W | M | N | F | throat | Cough | ~° F. | Ache | Used* |
| 1 | 06:15 | y | | | | | | | | | 123456 |
| 2 | | | | | | | | | | | |
| 3 | 08:30 | y | y | | y | y | | | 99 | | 123456 |
| 4 | 09:45 | y | y | | y | y | | | 101 | | 123456 |
| 5 | 10:30 | y | | | y | | | | 98 | | 123456 |
| 6 | | | | | | | | | | | |
| 7 | | | | | | | | | | | |
| 8 | | | | | | | | | | | |
| 9 | | | | | | | | | | | |
| 10 | | | | | | | | | | | |
| 11 | | | | | | | | | | | |
| 12 | 17:00 | | | | y | | | | | | 13456 |
| 13 | | | | | | | | | | | |
| 14 | 19;00 | | | | | | | | | | 13456 |
| 15 | 21:00 | | | | | | | | | | 13456 |
| 16 | | | | | | | | | | | |
| 17 | | | | | | | | | | | |
| 18 | | | | | | | | | | | |
| 19 | | | | | | | | | | | |
| 20 | | | | | | | | | | | |
| 21 | | | | | | | | | | | |
| 22 | | | | | | | | | | | |
| 23 | | | | | | | | | | | |
| 24 | | | | | | | | | | | |

*1. Pint of Water; 2. Cold Eeze lozenge; 3. Three Zarbees pills; 4. Elderberry capsule; 5. Two Garlic tabs; 6. Two Echinacea
Early symptoms: ST = scratchy throat, TW = temperature wobble, CM = cranky mood, SN = snotty nose, SF = sickish feeling
For each hour show by numerical ID the products used. Record below any issues, or special results. Circle if in contact: COLDS, FLU, or COVID19. If symptoms persist beyond Hour 4, act as though you have something serious. Continue treatments approximately as follow: Hour 1: 123456; Hour 2: 123456; Hour 3: 123456; Hour 4; 123456: Hour 8: 13456 Hour 9: 13456 Hour 10: 13456; Hour 11: 13456 (Note: zinc lozenge is left out starting at Hour 8). If symptoms persist beyond 24 hours, record results on a 2^{nd} copy of this form, and get medical help if necessary. If you take prescription drugs, talk to your doctor about contraindications. For example, zinc can interfere with thyroid medication.

TABLE 5
False alarm at Hour 1, military time 17:30 with an insurance treatment at 19:30
is resolved by 19:00 with precautionary treatment at 21:00
URV2020 24-Hr Symptom-Data-Trainer
Date _ _: _ _ : _ _ _ _, Time of onset _ _: _ _ am/pm (circle one)
Zip Code _____; Age___: coded ID ____: Estimate health prior to onset ________.
Use ~° F. for Fever, Y for Yes, and a Blank for No. The chart starts at Hour One of
suspected disease onset. Record results for each of the following 23 hours after that.
Use new forms for any reports after hour 24. Share below known cold, flu, or
COVID19 contacts + any events not included in the chart.

| Hour + | | | | | | | | | Fever | | |
|---|---|---|---|---|---|---|---|---|---|---|---|
| military | | S | T | C | S | S | Sore | | in | | Remedy |
| Time | | T | W | M | N | F | throat | Cough | ~° F. | Ache | Used* |
| 1 | 17:30 | Y | Y | | | | | | | | 123456 |
| 2 | | | | | | | | | | | |
| 3 | 19:30 | | | | | | | | | | 123456 |
| 4 | | | | | | | | | | | |
| 5 | | | | | | | | | | | |
| 6 | | | | | | | | | | | |
| 7 | | | | | | | | | | | |
| 8 | | | | | | | | | | | |
| 9 | | | | | | | | | | | |
| 10 | | | | | | | | | | | |
| 11 | | | | | | | | | | | |
| 12 | | | | | | | | | | | |
| 13 | | | | | | | | | | | |
| 14 | | | | | | | | | | | |
| 15 | | | | | | | | | | | |
| 16 | | | | | | | | | | | |
| 17 | | | | | | | | | | | |
| 18 | | | | | | | | | | | |
| 19 | | | | | | | | | | | |
| 20 | | | | | | | | | | | |
| 21 | | | | | | | | | | | |
| 22 | | | | | | | | | | | |
| 23 | | | | | | | | | | | |
| 24 | | | | | | | | | | | |

*1. Pint of Water; 2. Cold Eeze lozenge; 3. Three Zarbees pills; 4. Elderberry capsule; 5. Two Garlic tabs; 6. Two Echinacea
Early symptoms: ST = scratchy throat, TW = temperature wobble, CM = cranky mood, SN = snotty nose, SF = sickish feeling
For each hour show by numerical ID the products used. Record below any issues, or special results. Circle if in contact: COLDS, FLU, or COVID19. If symptoms persist beyond Hour 4, act as though you have something serious. Continue treatments approximately as follow: Hour 1: 123456; Hour 2: 123456; Hour 3: 123456; Hour 4; 123456: Hour 8: 13456 Hour 9: 13456 Hour 10: 13456; Hour 11: 13456 (Note: zinc lozenge is left out starting at Hour 8). If symptoms persist beyond 24 hours, record results on a 2^{nd} copy of this form, and get medical help if necessary. If you take prescription drugs, talk to your doctor about contraindications. For example, zinc can interfere with thyroid medication.

Example 7

### Determining the Efficacy of the URV2020 Method and Corresponding Kit

To determine the efficacy of the URV2020 method and corresponding kit, a double blind test is performed. Numerically labelled kits are provided to asymptomatic but SARS-CoV-2 infected individuals. 50% of the kits possess the ingredients and instructions to perform the URV2020 method (URV2020 kit). 50% of the kits provided are a placebo, in which the means to perform the URV2020 method are not provided. The use of an approximately equal number of placebo and URV2020 kits automatically divides the test subjects into two large, randomly selected groups of individuals. With data now showing that 40% of individuals tested for SARS-CoV-2 are positive, asymptomatic carriers, there is no problem in creating two large, randomly selected, positively testing, asymptomatic groups. The individuals are tested for SARS-CoV-2 infection just prior to receipt of a kit. The individuals are tested daily for SARS-CoV-2 until all test subjects test have tested negative for SARS-CoV-2. In order to determine the efficacy the URV2020 method and corresponding URV2020 kit, the length of time between the test immediately preceding receipt of the kit and a negative test is determined. If individuals that receive the URV2020 kit are testing negative for SARS-CoV-2 sooner than the placebo group on a significant, statistical average, the methods of the subject invention are effective.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A method for treating or preventing an upper respiratory viral infection, or a symptom thereof, in a human subject, said method comprising administering to the subject an initial Round 1 treatment comprising:
i) First, drinking 8 to 16 ounces of water rapidly;
ii) Second, dissolving in the mouth about 18.3 mg of zinc, together with elderberry fruit extract blend, about 300 μg vitamin A, about 500 mg vitamin C, about 20 μg vitamin D₃, about 15 mg vitamin E, about 1.7 mg vitamin B₆, about 2.4 μg vitamin B₁₂, about 666 μg folate, and about 15 μg selenium; and
iii) Third, swallowing with a liquid about 800 mg Echinacea, about 4000 mg garlic allicin, and about 335 mg elderberry fruit extract after ingredients of step ii) have dissolved.

2. The method of claim 1, wherein, if after the initial treatment of Round 1, a symptom of viral infection persists, the method further comprises:
iv) Fourth, repeating steps i), ii) and iii) of Round 1 one-time, two-times, or three-times within about three hours to about six hours of the initial step i).

3. The method of claim 2, wherein, if after step iv) a symptom of viral infection persists, the method further comprises administering an initial Round 2 treatment, approximately 4 hours to approximately 18 hours after the initial Round 1, the initial Round 2 treatment comprising:
v) First, drinking a pint of water rapidly;
vi) Second, dissolving in the mouth about 5 mg of zinc, elderberry fruit extract blend, about 300 μg vitamin A, about 500 mg vitamin C, about 20 μg vitamin D₃, about 15 mg vitamin E, about 1.7 mg vitamin B₆, about 2.4 μg vitamin B₁₂, about 666 μg folate, and about 15 μg selenium; and
vii) Third, swallowing about 800 mg Echinacea, about 4000 mg garlic allicin, and about 335 mg elderberry fruit extract after ingredients of step vi) have dissolved.

4. The method of claim 3, wherein, if after the initial treatment of Round 2, a symptom of viral infection persists, the method further comprises:
viii) Fourth, repeating steps v), vi) and vii) of Round 2 one-time, two-times, or three-times within about three hours to about six hours of the initial step v).

5. The method of claim 1, wherein the zinc administered in each Round 1 treatment is administered as one 13.3 mg of zinc Cold Eeze lozenge and three Zarbee's Elderberry and honey tablets that together comprise an additional 5 mg of zinc; the elderberry fruit extract blend is administered as part of a 326 mg blend in the three Zarbee's Elderberry and honey tablets; the vitamin A, vitamin C, vitamin D₃, vitamin E, vitamin B₆, vitamin B₁₂, folate, and selenium is administered as parts of the three Zarbee's Elderberry and honey tablets; the Echinacea is administered as two 400 mg Nature's Bounty Echinacea capsules; the garlic allicin is administered as two 2000 mg Nature's Bounty Garlic capsules; and the elderberry fruit extract is administered as one New Chapter Elderberry Force capsule.

6. The method of claim 3, wherein the zinc administered in each Round 2 treatment is administered as three Zarbee's Elderberry and honey tablets that in total comprise 5 mg of zinc; the elderberry fruit extract blend is administered as part of a 326 mg blend in the three Zarbee's Elderberry and honey tablets; the vitamin A, vitamin C, vitamin D₃, vitamin E, vitamin B₆, vitamin B₁₂, folate, and selenium is administered as parts of the three Zarbee's Elderberry and honey tablets; the Echinacea is administered as two 400 mg Nature's Bounty Echinacea capsules; the garlic allicin is administered as two 2000 mg Nature's Bounty Garlic capsules; and the elderberry fruit extract is administered as one New Chapter Elderberry Force capsule.

7. The method of claim 1, wherein Round 1 is initiated within from immediately upon the subject noticing a first symptom or having contact with a person who presents a viral infection symptom or is infected with a respiratory virus to within about 24 hours of the noticing of the first symptom or within about 48 hours of the contact with the person that presents a viral infection symptom and/or is infected with a respiratory virus.

8. The method of claim 7, wherein, if after completing the initial Round 1 treatment there is an assessment of no indication of any symptom continuing, administering to the subject a second Round 1 treatment within an hour or two of that assessment as a precautionary treatment.

9. The method of claim 3, wherein the initial Round 2 treatment is initiated within 1 to 14 hours of a 4^{th} Round 1 treatment and, if there has been an assessment of the subject having no continuance of symptoms after the 4^{th} Round 1 treatment of after the initial Round 2 treatment, administering a second Round 2 treatment within 2 to 4 hours of the initial Round 2 treatment as a precautionary treatment.

10. The method of claim 1, wherein the upper respiratory viral infection is caused by an influenza virus, respiratory syncytial virus (RSV), parainfluenza virus (NV), human metapneumovirus (hMPV), rhinovirus, or coronavirus (CoV).

11. The method of claim 1, wherein the human subject has the upper respiratory virus infection at the time of said administering.

12. The method of claim 1, wherein the human subject has previously had the upper respiratory virus infection at the time of said administering.

13. The method of claim 1, wherein the human subj ect does not have the upper respiratory virus infection at the time of said administering.

14. The method of claim 1, wherein the human subject is aware of having interacted with an individual known to have at least one upper respiratory viral infection symptom.

15. A composition of matter, comprising about 18.3 mg of zinc, elderberry fruit extract blend, about 300 μg vitamin A, about 500 mg vitamin C, about 20 μg vitamin D₃, about 15 mg vitamin E, about 1.7 mg vitamin B₆, about 2.4 μg vitamin B₁₂, 666 μg folate, about 15 μg selenium, about 800 mg Echinacea, about 4000 mg garlic allicin, and about 335 mg elderberry fruit extract.

16. A method for treating or preventing an upper respiratory viral infection, or a symptom thereof, in a human subject, said method comprising administering to the subject an initial Round 1 treatment comprising:
i) First, drinking 8 to 16 ounces of water rapidly and swallowing with the water about 800 mg Echinacea, about 4000 mg garlic allicin, and about 335 mg elderberry fruit extract; and
ii) Second, dissolving in the mouth about 18.3 mg of zinc, together with elderberry fruit extract blend, about 300 μg vitamin A, about 500 mg vitamin C, about 20 μg vitamin D₃, about 15 mg vitamin E, about 1.7 mg vitamin B₆, about 2.4 μg vitamin B₁₂, about 666 μg folate, and about 15 μg selenium.

17. The method of claim 16, wherein, if after the initial treatment of Round 1, a symptom of viral infection persists, the method further comprises:
iii) Third, repeating steps i) and ii) of Round 1 one-time, two-times, or three-times within about three hours to about six hours of the initial step i).

18. The method of claim 17, wherein, if after step iii) a symptom of viral infection persists, the method further comprises administering an initial Round 2 treatment, approximately 4 hours to approximately 18 hours after the initial Round 1, the initial Round 2 treatment comprising:
iv) First, drinking 8 to 16 ounces of water rapidly and swallowing with the water about 800 mg Echinacea, about 4000 mg garlic allicin, and about 335 mg elderberry fruit extract; and
v) Second, dissolving in the mouth about 5 mg of zinc, elderberry fruit extract blend, about 300 μg vitamin A, about 500 mg vitamin C, about 20 μg vitamin D₃, about 15 mg vitamin E, about 1.7 mg vitamin B₆, about 2.4 μg vitamin B₁₂, about 666 μg folate, and about 15 μg selenium.

19. The method of claim 18, wherein, if after the initial treatment of Round 2, a symptom of viral infection persists, the method further comprises:
vi) Fourth, repeating steps iv) and v) of Round 2 one-time, two-times, or three-times within about three hours to about six hours of the initial step iv).

20. An article of manufacture, comprising:
(a) packaged dosage formulation comprising 18.3 mg of zinc, elderberry fruit extract blend, about 300 μg vitamin A, about 500 mg vitamin C, about 20 μg vitamin D₃, about 15 mg vitamin E, about 1.7 mg vitamin B₆, about 2.4 μg vitamin B₁₂, 666 μg folate, about 15 μg selenium, about 800 mg Echinacea, about 4000 mg garlic allicin, and about 335 mg elderberry fruit extract, in a pharmaceutically acceptable dosage in one or more packages, packets, or containers; and instructions for administering the 18.3 mg of zinc, elderberry fruit extract blend, about 300 μg vitamin A, about 500 mg vitamin C, about 20 μg vitamin D₃, about 15 mg vitamin E, about 1.7 mg vitamin B₆, about 2.4 μg vitamin B₁₂, 666 μg folate, about 15 μg selenium, about 800 mg Echinacea, about 4000 mg garlic allicin, and about 335 mg elderberry fruit extract; or
(b) a kit comprising, in one or more containers, 18.3 mg of zinc, elderberry fruit extract blend, about 300 μg vitamin A, about 500 mg vitamin C, about 20 μg vitamin D₃, about 15 mg vitamin E, about 1.7 mg vitamin B₆, about 2.4 μg vitamin B₁₂, 666 μg folate, about 15 μg selenium, about 800 mg Echinacea, about 4000 mg garlic allicin, and about 335 mg elderberry fruit extract; and instructions for administering the 18.3 mg of zinc, elderberry fruit extract blend, about 300 μg vitamin A, about 500 mg vitamin C, about 20 μg vitamin D₃, about 15 mg vitamin E, about 1.7 mg vitamin B₆, about 2.4 μg vitamin B₁₂, 666 μg folate, about 15 μg selenium, about 800 mg Echinacea, about 4000 mg garlic allicin, and about 335 mg elderberry fruit extract with instructions to treat or prevent a respiratory virus infection.

21. The method of claim 1, wherein the human subject is symptomatically infected by the upper respiratory virus.

22. The method of claim 1, wherein the human subject is asymptomatically infected by the upper respiratory virus.

23. The method of claim 16, wherein the human subject is symptomatically infected by the upper respiratory virus.

24. The method of claim 16, wherein the human subject is asymptomatically infected by the upper respiratory virus.
